SEQUENCE LISTING (16 sequences)

SEQ ID NO: 1 - protein, 221 aa, Artificial Sequence
  Light chain of Ab-REW
DIQMTQSPSSLSASVGDRVTIKCQASQSIFSGLAWYQQKPGKAPKLLIYGASKLASGVPSRFSGSGSGTEFTLTISGLQPADFATYYCQSYYYSSSSSDGSYAFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 452 aa, Artificial Sequence
  Heavy chain of a humanized anti-TNFalpha antibody
  MISC_FEATURE (452)..(452): Xaa is Lysine or missing (absent)
EVQLVESGGGLVQPGGSLRLSCKASGIDFNNYGIGWVRQAPGKGLEWITYIYPGFAITNFANSVKGRFTISSDNSKNTVYLQMNSLRAEDTAVYYCARDPVYATSSGYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 3 - protein, 11 aa, Artificial Sequence
  CDR L1 of antibody Ab-REW
QASQSIFSGLA

SEQ ID NO: 4 - protein, 7 aa, Artificial Sequence
  CDR L2 of antibody Ab-REW
GASKLAS

SEQ ID NO: 5 - protein, 15 aa, Artificial Sequence
  CDR L3 of antibody Ab-REW
QSYYYSSSSSDGSYA

SEQ ID NO: 6 - protein, 10 aa, Artificial Sequence
  CDR H1 of antibody Ab-REW
GIDFNNYGIG

SEQ ID NO: 7 - protein, 17 aa, Artificial Sequence
  CDR H2 of antibody Ab-REW
YIYPGFAITNFANSVKG

SEQ ID NO: 8 - protein, 13 aa, Artificial Sequence
  CDR H3 of antibody Ab-REW
DPVYATSSGYFDL

SEQ ID NO: 9 - protein, 122 aa, Artificial Sequence
  VH of antibody Ab-REW
EVQLVESGGGLVQPGGSLRLSCKASGIDFNNYGIGWVRQAPGKGLEWITYIYPGFAITNFANSVKGRFTISSDNSKNTVYLQMNSLRAEDTAVYYCARDPVYATSSGYFDLWGQGTLVTVSS

SEQ ID NO: 10 - protein, 114 aa, Artificial Sequence
  VL of antibody Ab-REW
DIQMTQSPSSLSASVGDRVTIKCQASQSIFSGLAWYQQKPGKAPKLLIYGASKLASGVPSRFSGSGSGTEFTLTISGLQPADFATYYCQSYYYSSSSSDGSYAFGGGTKLTVLG

SEQ ID NO: 11 - protein, 451 aa, Artificial Sequence
  Heavy chain of antibody Ab-REW
EVQLVESGGGLVQPGGSLRLSCKASGIDFNNYGIGWVRQAPGKGLEWITYIYPGFAITNFANSVKGRFTISSDNSKNTVYLQMNSLRAEDTAVYYCARDPVYATSSGYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVEHEALHWHYTQKSLSLSPG

SEQ ID NO: 12 - protein, 233 aa, Artificial Sequence
  Light chain of antibody cV1q-huFc
MSVPTQVLGLLLLWLTDARCNIQLTQSPSLLSASVGDRVTLSCKGSQNINNFLAWYQQELGEAPKLLIYNTNSLQTGIPSRFTGSGSGTDYTLTISSLQPEDVATYFCYQYNNGNTFGVGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 467 aa, Artificial Sequence
  Heavy chain of antibody cV1q-huFc
MEWSWVFLFFLSVTTGVHSQVQLKESGPGLVQPSQTLSLTCTVSGFSLTSYNVHWVRQPPGKGLEWMGRMRYNGDTSYNSALKSRLSISRDTSKNQVFLKMNSLQTDDTGTYYCTRDRFSWASYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVEHEALHWHYTQKSLSLSPG

SEQ ID NO: 14 - protein, 452 aa, Artificial Sequence
  Heavy chain of a variant of antibody Ab-REW
EVQLVESGGGLVQPGGSLRLSCKASGIDFNNYGIGWVRQAPGKGLEWITYIYPGFAITNFANSVKGRFTISSDNSKNTVYLQMNSLRAEDTAVYYCARDPVYATSSGYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVEHEALHWHYTQKSLSLSPGK

SEQ ID NO: 15 - protein, 451 aa, Artificial Sequence
  Heavy chain of antibody Ab-AA
EVQLVESGGGLVQPGGSLRLSCKASGIDFNNYGIGWVRQAPGKGLEWITYIYPGFAITNFANSVKGRFTISSDNSKNTVYLQMNSLRAEDTAVYYCARDPVYATSSGYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVAWESNGQPENNYKTTPPVLDSDGSFFLYSKLTVDKSRWQQGNVFSCSVMHEALHAHYTQKSLSLSPG

SEQ ID NO: 16 - protein, 452 aa, Artificial Sequence
  Modified heavy chain of antibody Ab-AA
EVQLVESGGGLVQPGGSLRLSCKASGIDFNNYGIGWVRQAPGKGLEWITYIYPGFAITNFANSVKGRFTISSDNSKNTVYLQMNSLRAEDTAVYYCARDPVYATSSGYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVAWESNGQPENNYKTTPPVLDSDGSFFLYSKLTVDKSRWQQGNVFSCSVMHEALHAHYTQKSLSLSPGK

(19) United States
(12) Patent Application Publication
(10) Pub. No.: US 2022/0119515 A1
(43) Pub. Date: Apr. 21, 2022

(54) TOPICAL TREATMENT OF IMMUNE CHECKPOINT INHIBITOR INDUCED DIARRHOEA, COLITIS OR ENTEROCOLITIS USING ANTIBODIES AND FRAGMENTS THEREOF

(71) Applicant: Tillotts Pharma AG, Rheinfelden (CH)

(72) Inventors: Esther Maria FURRER, Rheinfelden (CH); Felipe VARUM, Riehen (CH); Roberto BRAVO, Rheinfelden (CH); Johannes SPLEISS, Rheinfelden (CH); Marijana NEDELJKOVIC POTIC, Lausanne (CH); Ortrud GERSTNER, Rheinfelden (CH); Cristina BRUNO, Rheinfelden (CH)

(73) Assignee: Tillotts Pharma AG, Rheinfelden (CH)

(21) Appl. No.: 17/298,337

(22) PCT Filed: Dec. 6, 2019
(86) PCT No.: PCT/EP2019/083992

§ 371 Date: May 28, 2021

(30) Foreign Application Priority Data
Dec. 7, 2018 (EP) PCT/EP2018/084057

(51) Int. Cl. C07K 16/24; A61K 9/50; A61P 1/14; A61K 9/14

(52) U.S. Cl. CPC C07K 16/241; A61K 9/5026; A61K 9/1623; A61K 9/145; A61P 1/14

(57) ABSTRACT

The present invention relates to the therapeutic topical use of compositions containing antibody molecules or functional fragments or derivatives specific to tumour necrosis factor alpha (TNFα), for treating or preventing immune checkpoint (ICP) inhibitor-induced adverse events.

### PRIORITY

This application corresponds to the U.S. National phase of International Application No. PCT/EP2019/083992, filed Dec. 6, 2019, which, in turn, claims priority to International Patent Application No. PCT/EP2018/084057 filed Dec. 7, 2018, the contents of which are incorporated by reference herein in their entirety.

### SEQUENCE LISTING

The instant application contains a Sequence Listing that has been submitted in ASCII format via EFS-Web and is hereby incorporated by reference in its entirety. Said ASCII copy, created on Apr. 15, 2021, is named LNK_230 US_SEQ_LIST.txt and is 32,144 bytes in size.

### FIELD OF THE INVENTION

The present invention relates to the therapeutic use of compositions containing antibody molecules and functional fragments and derivatives specific to tumour necrosis factor alpha (TNFα), in the topical treatment of at least one adverse event of the gastro-intestinal tract induced by a cancer therapy in a patient, preferably induced by one or more immune checkpoint (ICP) inhibitors. In particular, the invention pertains to the therapeutic use of such compositions in the topical treatment of immune checkpoint (ICP) inhibitor-induced diarrhoea, colitis and/or enterocolitis, in particular in patients, who have not stopped taking ICP inhibitors as cancer treatment; as well as to the use of such compositions as an at least partially prophylactic or therapy for ICP inhibitor-induced diarrhoea, colitis and/or enterocolitis in patient undergoing treatment with ICP inhibitors.

### BACKGROUND OF THE INVENTION

In recent years the use of inhibitors targeting so-called immune checkpoints (ICPs) and thereby activating the immune system against cancer cells has emerged as a promising new cancer therapy strategy.

ICP inhibitors, which are immune-stimulatory agents that ‘unblock’ an existing immune response or which unblock the initiation of an immune response, have been shown to be very effective at treating certain cancer types. The ICP members that have received the most attention in recent years in the search for ICP inhibitors are programmed death-1 (PD-1), programmed death-ligand 1 (PD-L1) and cytotoxic T-lymphocyte-associated protein-4 (CTLA-4). PD-1, PD-L1 and CTLA-4 inhibitors, for example have shown significant potential in improving overall survival in patients with malignant melanoma, malignant non-small cell lung carcinoma, squamous cell cancers of the head and neck and renal cell carcinoma (Wang et al., Inflamm Bowel Dis, 24(8):1695-1705, July 2018). Moreover, also in patients with malignant lymphoma ICP inhibitors have shown promising results in phase I and II clinical trials and PD-1 inhibitor nivolumab was approved for the treatment of relapsed or refractory classical Hodgkin lymphoma by the Food and Drug Administration in May 2016 (Hude et al., Haematologica, 2017 January, 102(1): 30-42).

CTLA-4, PD-1 and its ligands are members of the B7-CD28 family of co-signalling molecules that play important roles throughout all stages of T-cell function and other cell functions. The PD-1 receptor is expressed on the surface of activated T-cells (and B cells) and, under normal circumstances, binds to its ligands (PD-L1 and PD-L2) that are expressed on the surface of antigen-presenting cells, such as dendritic cells or macrophages. This interaction sends a signal into the T-cell and essentially switches it off/inhibits it.

Currently available ICP inhibitors, which target pathways that inhibit cytotoxic T-cell activation, proliferation, and function, include antibodies specific to CTLA-4, PD-1 and PD-L1. For example monoclonal antibodies that target either PD-1 or PD-L1 can block this binding and increase the immune response against cancer cells. These inhibitors have shown to be very promising in treating a range of cancer types.

Whereas the augmentation of a patients' native adaptive immune systems by ICP inhibitors leads to improved tumour control, nonspecific immune activation often results in the development of immune-related adverse events affecting a variety of organ systems, and in particular the gastrointestinal (GI) tract, like ICP inhibitor-induced colitis, enterocolitis and/or diarrhoea. In fact, findings from clinical trials show that the most common severe or life-threatening (grades 3 and 4) immune-related adverse events occur in the GI tract (Wang et al., Inflamm Bowel Dis, 24(8):1695-1705, July 2018; Wang D Y, et al. Fatal Toxic Effects Associated With Immune Checkpoint Inhibitors. A Systematic Review and Meta-analysis. JAMA Oncol. Published online Sep. 13, 2018. doi:10.1001/jamaoncol.2018.3923).

For example immune-related adverse events involving the GI tract, such as ICP inhibitor-induced colitis, have been reported in about 21% to 44% of patients treated with CTLA-4 inhibitors and a bit less frequently in patients treated with PD-1/PD-L1 inhibitors (Wang et al., Inflamm Bowel Dis, 24(8):1695-1705, July 2018).

Colitis is as a disorder characterized by inflammation of the large intestine/colon. Enteritis is defined as a disorder characterized by inflammation of the small intestine. Enterocolitis is as a disorder characterized by inflammation of both the small intestine and the large intestine/colon, i.e. a combination of enteritis and colitis. Symptoms characterizing ICP inhibitor-induced colitis include diarrhoea, abdominal pain, nausea, cramping, blood or mucus in stool or changes in bowel habits, fever, abdominal distention, obstipation and constipation (Brahmer et al., Management of Immune-Related Adverse Events in Patients Treated With Immune Checkpoint Inhibitor Therapy: American Society of Clinical Oncology (ASCO) Practice Guideline, J Clin Oncol. 2018 Jun. 10; 36(17):1714-1768). Symptoms characterizing ICP inhibitor-induced enterocolitis include the symptoms listed above for ICP inhibitor-induced colitis.

As described in the ASCO Practice Guideline for the Management of Immune-Related Adverse Events in Patients Treated with Immune Checkpoint Inhibitor Therapy, the severity of ICP inhibitor-induced colitis clinically can be graded into four grades based on the National Cancer Institute's CTCAE for diarrhoea, version 4. Grade 1 is characterized by diarrhoea with <4 stools per day over baseline; and mild increase in ostomy output compared with baseline. Grade 2 is characterized by diarrhoea with 4-6 stools per day over baseline; and moderate increase in ostomy output compared with baseline. Grade 3 is characterized by diarrhoea with 7 stools per day over baseline; incontinence; severely increased volume of stool; and severe increase in ostomy output compared with baseline; and hospitalization is indicated. Grade 4 is characterized by diarrhoea with >7 stools per day over baseline; overall the same symptoms as grade 3 but with life-threatening consequences, like perforations, ileus and/or fever; and hospitalization is usually necessary (Brahmer et al., Management of Immune-Related Adverse Events in Patients Treated With Immune Checkpoint Inhibitor Therapy: American Society of Clinical Oncology Practice Guideline, J Clin Oncol. 2018 Jun. 10; 36(17):1714-1768). An equivalent grading can be applied to patients suffering from ICP inhibitor-induced colitis or enterocolitis.

Treatment for ICP inhibitor-induced colitis and enterocolitis is typically based on symptom severity, involving immunosuppression with systemically administered corticosteroids for moderate to severe symptoms, and in steroid-refractory cases, systemic application of biologics such as infliximab.

The ASCO Practice Guideline recommends for grade 2 toxicities associated with ICP inhibitor-induced colitis administration of corticosteroids, unless diarrhoea is transient, starting with initial dose of 1 mg/kg/day prednisone or equivalent. During therapy with corticosteroids treatment with ICP inhibitors should be stopped. When symptoms improve to grade 1 or less, corticosteroids should be tapered over at least 4 to 6 weeks before resuming treatment with ICP inhibitors.

For grade 3 toxicities the guideline recommends administration of corticosteroids starting with initial dose of 1 to 2 mg/kg/day prednisone or equivalent. It should be considered to permanently discontinuing CTLA-4 agents, while PD-1 and PD-L1 agents may be restarted, if the patient recovers to grade 1 or less. If symptoms persist for more than 3 to 5 days or recur after improvement, administration of intravenous corticosteroids or non-corticosteroid immunosuppressant (e.g. infliximab) should be considered.

For grade 4 toxicities the guideline recommends intravenous administration of corticosteroids at a dose of 1 to 2 mg/kg/day (methyl)prednisolone or equivalent until the symptoms improve to grade 1, to then start tapering corticosteroids over 4 to 6 weeks. ICP inhibitors should be permanently discontinued. If symptoms are refractory within 2 to 3 days, administration of 5-10 mg/kg infliximab (e.g. Infliximab 5 mg/kg every 2 weeks) should be considered. Guidance on the clinical management of ICP inhibitor toxicities is also provided in Hryniewicki et al. J Emerg Med. 2018 October; 55(4):489-502.

Thus, persistent grade 2 ICP inhibitor-induced colitis, and almost any grade 3/4 ICP inhibitor-induced colitis will typically require management with systemic corticosteroids. The use of prophylactic steroids (e.g. oral budesonide) has not been shown to be effective at treating the development of diarrhoea or colitis caused by ICP inhibitors. In patients refractory to treatment with systemic corticosteroids, systemic treatment with a non-corticosteroid like the anti-TN Fa specific antibody infliximab is recommended. According to the ASCO Practice Guideline, enteritis, and consequently also enterocolitis in general, can be managed similar as colitis, including corticosteroid and/or infliximab.

Corticosteroids have several side-effects, especially in prolonged use and at high doses. In particular, patients treated with corticosteroids often have an increased susceptibility to infection. Moreover, the use of systemic corticosteroids, in addition to higher infection rate, results in other potential side-effects including osteoporosis; fractures; osteonecrosis; increased cardiovascular risk; gastritis, peptic ulcer disease; worsening of diabetes; nervousness or restlessness; high blood pressure; sleeplessness; water retention and swelling; cataracts or glaucoma; muscle weakness; sudden mood swings; easy bruising; and weight gain. Therefore, long-term systemic treatment with corticosteroids should be avoided.

Furthermore, systemic treatment with corticosteroids at higher doses (e.g. 1-2 mg/kg/day prednisone or equivalent) requires interruption (or permanent discontinuation) of treatment with ICP inhibitors. Such an interruption compromises the anti-cancer effect of these ICP inhibitors. Therefore, it would be preferable to avoid such interruptions.

Non-steroidal agents having an immunosuppressive effect, e.g. anti-TNFα antibodies, do not share many of the adverse side-effects of systemic treatment with corticosteroids even though, also anti-TNFα antibodies, like infliximab, are usually administered systemically. Additionally, infection rates have been reported to be numerically higher among patients suffering from ICP inhibitor-induced colitis, who received systemic corticosteroids for longer durations, compared to administration of systemic anti-TNFα antibodies. Therefore, early non-steroid immunosuppressive therapy may ensure a more favourable overall outcome (Wang et al. J ImmunoTher Cancer (2018) 6:37).

At present, the standard therapy for the treatment of severe ICP inhibitor-induced colitis or enterocolitis using anti-TNFα specific antibodies involves the regular systemic administration of an anti-TNFα antibody by intravenous infusion. However, also systemic administration with anti-TNFα antibodies in cancer patients requires interruption of any ICP inhibitor treatment, thereby compromising the successful cancer treatment with the ICP inhibitor.

Moreover, intravenous administration can give rise to complications including acute infusion reaction, hypersensitivity and anaphylactic shock. Additionally, systemic application of anti-TNFα antibodies bears a multitude of risks associated with systemic inhibition of the immune defence function of TNFα in the patient, including for example infectious complications. In addition, immunogenicity to systemically applied antibodies may lead to their neutralisation, resulting in a secondary loss of response. Finally, melanoma and Merkel cell carcinoma have been reported in patients treated with TNF blocker therapy, including infliximab.

Thus, at present the treatment of ICP inhibitor-induced adverse events in the GI tract, like ICP inhibitor-induced colitis, enterocolitis and/or diarrhoea, with moderate to severe symptoms requires interruption of the ICP inhibitor treatment and systemic application of a, usually steroidal, immunosuppressant. This is detrimental to the effectiveness of the cancer therapy and results in undesirable side-effects on a systemic level and in the case of melanoma, recurrence of the underlying disease.

There exists a need for an alternative treatment of ICP inhibitor-induced adverse events of the GI tract such as ICP inhibitor-induced diarrhoea, colitis or enterocolitis allowing for a specific targeting of inflamed tissues in the GI tract, and in particular in the ileum and the large intestine, without the need for interrupting treatment with ICP inhibitors. The treatment should particularly enable the effective targeting of the tissue affected by the inhibitor-induced adverse event in the GI tract, without requiring systemic application. In addition, there is also a need for a prophylactic treatment to prevent, or delay the onset of, adverse effects affecting the GI tract caused by treatment with ICP inhibitors; in particular for an early treatment to prevent serious complications like ileus and perforation as well as need for urgent endoscopies, hospital admissions and surgery.

### SUMMARY OF THE INVENTION

Surprisingly it has been found by the present inventors that by topically treating immune-related adverse events induced by ICP inhibitors in the GI tract, thereby removing the need for systemic immunosuppressive treatment, this overcomes the necessity to stop treatment with ICP inhibitors by allowing the concomitant topical treatment in the GI with immunosuppressive TNFα-specific antibodies or functional fragments thereof and systemic treatment with ICP inhibitors, thereby avoiding compromising the successful cancer treatment with the ICP inhibitors. Despite the fact that ICP inhibitors for treating cancer have been known for more than 15 years, it has never been suggested that TNFα-specific antibodies could be topically administered to treat or prevent the immune-related adverse effects of the ICP inhibitor therapy.

The present invention provides a composition for use in the topical treatment in the ileum and/or the large intestine of ICP inhibitor-induced diarrhoea, colitis or enterocolitis. Moreover, the present invention provides a composition for use in the topical treatment in the ileum and/or the large intestine as a prophylactic therapy for ICP inhibitor-induced diarrhoea, colitis or enterocolitis. In particular, the present invention provides a composition for use in an early therapy for ICP inhibitor induced diarrhoea to prevent more severe adverse events affecting the gastrointestinal tract, such as colitis or enterocolitis.

The present invention therefore relates to the subject matter defined in the following items 1 to 69:

[1] A pharmaceutical composition comprising an active agent selected from the group consisting of antibodies specific to tumour necrosis factor alpha (TNFα) and functional fragments and derivatives thereof, for use in the treatment or prevention of at least one adverse event of the gastro-intestinal tract induced by a cancer therapy in a patient, preferably induced by one or more immune checkpoint (ICP) inhibitors, wherein said treatment or prevention comprises the topical administration of said composition to the affected part of the gastrointestinal tract of said patient, e.g. to the ileum and/or the large intestine of said patient; or

A pharmaceutical composition comprising an active agent selected from the group consisting of antibodies specific to tumour necrosis factor alpha (TNFα) and functional fragments and derivatives thereof, for use in the treatment or prevention of at least one adverse event of the gastro-intestinal tract induced by a cancer therapy in a patient, preferably induced by one or more immune checkpoint (ICP) inhibitors, wherein said treatment or prevention comprises orally administering said composition to said patient.

[2] A pharmaceutical composition comprising an active agent selected from the group consisting of antibodies specific to tumour necrosis factor alpha (TNFα) and functional fragments and derivatives thereof, for use in preventing the progression or worsening of at least one adverse event of the gastro-intestinal tract induced by a cancer therapy in a patient, preferably induced by one or more immune checkpoint (ICP) inhibitors, wherein said prevention comprises the topical administration of said composition to the affected part of the gastrointestinal tract of said patient, e.g. to the ileum and/or the large intestine of said patient; or

A pharmaceutical composition comprising an active agent selected from the group consisting of antibodies specific to tumour necrosis factor alpha (TNFα) and functional fragments and derivatives thereof, for use in preventing the progression or worsening of at least one adverse event of the gastro-intestinal tract induced by a cancer therapy in a patient, preferably induced by one or more immune checkpoint (ICP) inhibitors, wherein said prevention comprises orally administering said composition to said patient.

[3] The composition for use according to item 1 or 2, which allows uninterrupted treatment, preferably systemic treatment, with one or more ICP inhibitors.

[4] The composition for use according to any one of items 1 to 3, wherein the patient is undergoing treatment, preferably systemic treatment, with one or more ICP inhibitors.

[5] The composition for use according to item 4, wherein treatment with one or more ICP inhibitors is interrupted for less than four weeks, preferably less than two weeks, more preferably less than one week, even more preferably less than five days, four days, three days, two days or one day, and most preferably wherein treatment is not interrupted.

[6] The composition for use according to any one of the preceding items, wherein the patient is suffering from, or at risk of developing, ICP inhibitor-induced colitis.

[7] The composition for use according to item 6, wherein said ICP inhibitor-induced colitis is characterized by grade 1 toxicity, and optionally wherein said treatment or prevention prevents progression to a higher grade toxicity.

[8] The composition for use according to item 6, wherein said ICP inhibitor-induced colitis is characterized by grade 2 toxicity, and optionally wherein said treatment or prevention prevents progression to a higher grade toxicity.

[9] The composition for use according to item 6, wherein said ICP inhibitor-induced colitis is characterized by grade 3 toxicity, and optionally wherein said treatment or prevention prevents progression to a higher grade toxicity.

[10] The composition for use according to item 6, wherein said ICP inhibitor-induced colitis is characterized by grade 4 toxicity.

[11] The composition for use according to any one of items 7 to 10, wherein said treatment or prevention prevents a discontinuation of the treatment with one or more ICP inhibitors.

[12] The composition for use according to any one of the preceding items, wherein the patient is suffering from, or at risk of developing, ICP inhibitor-induced enterocolitis.

[13] The composition for use according to item 12, wherein said ICP inhibitor-induced enterocolitis is characterized by grade 1 toxicity, and optionally wherein said treatment or prevention prevents progression to a higher grade toxicity.

[14] The composition for use according to item 12, wherein said ICP inhibitor-induced enterocolitis is characterized by grade 2 toxicity, and optionally wherein said treatment or prevention prevents progression to a higher grade toxicity.

[15] The composition for use according to item 12, wherein said ICP inhibitor-induced enterocolitis is characterized by grade 3 toxicity, and optionally wherein said treatment or prevention prevents progression to a higher grade toxicity.

[16] The composition for use according to item 12, wherein said ICP inhibitor-induced enterocolitis is characterized by grade 4 toxicity.

[17] The composition for use according to any one of items 13 to 16, wherein said treatment or prevention prevents a discontinuation of the treatment with one or more ICP inhibitors.

[18] The composition for use according to any one of the preceding items, wherein the patient is suffering from, or at risk of developing, ICP inhibitor-induced diarrhoea.

[19] The composition for use according to item 18, wherein said ICP inhibitor-induced diarrhoea is characterized by grade 1 toxicity, and optionally wherein said treatment or prevention prevents progression to a higher grade toxicity.

[20] The composition for use according to item 18, wherein said ICP inhibitor-induced diarrhoea is characterized by grade 2 toxicity, and optionally wherein said treatment or prevention prevents progression to a higher grade toxicity.

[21] The composition for use according to item 18, wherein said ICP inhibitor-induced diarrhoea is characterized by grade 3 toxicity, and optionally wherein said treatment or prevention prevents progression to a higher grade toxicity.

[22] The composition for use according to item 18, wherein said ICP inhibitor-induced diarrhoea is characterized by grade 4 toxicity.

[23] The composition for use according to any one of items 19 to 22, wherein said treatment or prevention prevents a discontinuation of the treatment with one or more ICP inhibitors.

[24] The composition for use according to any of the preceding items, wherein the composition is for use as a first-line treatment of a patient with ICP inhibitor-induced diarrhoea, colitis or enterocolitis; and/or wherein said patient is not being treated with corticosteroids; and/or wherein said patient has not been treated with corticosteroids.

[25] The composition for use according to any of the preceding items, wherein the composition is for use as second-line treatment in a patient with steroid-refractory ICP inhibitor-induced diarrhoea, colitis or enterocolitis.

[26] A pharmaceutical composition comprising an active agent selected from the group consisting of antibodies specific to TNFα and functional fragments and derivatives thereof, for use in the topical treatment in the ileum and/or the large intestine of a patient, as a prophylactic therapy for ICP inhibitor-induced diarrhoea, colitis or enterocolitis.

[27] The composition for use according to item 26, wherein the topical treatment prevents the development of grade 3 toxicities, preferably grade 2 toxicities, more preferably grade 1 toxicities, most preferably any symptoms, associated with ICP inhibitor-induced adverse events in the patient, preferably ICP inhibitor-induced diarrhoea, colitis or enterocolitis in the ileum and or the colon of the patient.

[28] The composition for use according to item 27, allowing at the same time treatment, preferably systemic treatment, with one or more ICP inhibitors.

[29] The composition for use according to items 26 to 28, wherein the patient is at the same time undergoing systemic treatment with one or more ICP inhibitors.

[30] The composition for use according to items 26 to 29, wherein the use of the composition prevents or minimizes and/or slows down symptoms associated with ICP inhibitor-induced diarrhoea, colitis or enterocolitis.

[31] The composition for use according to any of the preceding items, wherein the ICP inhibitor is capable of targeting an immune checkpoint selected from the group consisting of CTLA-4, PD-1, PD-L1, PDL2, B7-H3, B7-H4, BTLA, HVEM, TIM3, GAL9, LAG3, CSF-1R, VISTA, KIR, 2B4, CD160, CGEN-15049, CHK1, CHK2, A2aR, ADAR1, CD47, ICOS, TIGIT and the B-7 family of ligands.

[32] The composition for use according to any of the preceding items, wherein the one or more ICP inhibitors are selected from the group consisting of antibodies specific to cytotoxic T-lymphocyte-associated protein-4 (CTLA-4), antibodies specific to programmed cell death protein-1 (PD-1) and antibodies specific to programmed death-ligand 1 (PD-L1).

[33] The composition for use according to item 32, wherein the antibodies specific to PD-1 are selected from the group consisting of pembrolizumab, nivolumab, cemiplimab, and tislelizumab; the antibodies specific to PD-L1 are selected from the group consisting of atezolizumab, avelumab and durvalumab and the antibodies specific to CTLA-4 are selected from the group consisting of ipilimumab and tremelimumab.

[34] The composition for use according to item 32 or 33, wherein the one or more ICP inhibitors are a combination of antibodies specific to PD-1 and/or PD-L1 and antibodies specific to CTLA-4, preferably a combination of antibodies specific to PD-1 or PD-L1 and antibodies specific to CTLA-4.

[35] The composition for use according to any one of the preceding items, wherein the one or more ICP inhibitors modulate an immune response in the patient.

[36] The composition for use according to any one of the preceding items, wherein the one or more ICP inhibitors inhibit the growth of tumour cells in the patient.

[37] The composition for use according to any one of the preceding items, wherein the patient suffers from an infectious disease.

[38] The composition for use according to any one of the preceding items, wherein the patient is a cancer patient.

[39] The composition for use according to item 38, wherein the cancer is selected from the group consisting of melanoma; lymphoma, like classical Hodgkin lymphoma; glioma; urothelial carcinoma; Merkel cell carcinoma; renal cancer; head and neck squamous cell carcinoma, prostate cancer; breast cancer; colon cancer; and lung cancer.

[40] The composition for use according to item 39, wherein the cancer selected from the group consisting of bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the oesophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukaemia, acute myeloid leukaemia, chronic myeloid leukaemia, acute lymphoblastic leukaemia, chronic lymphocytic leukaemia, solid tumours of childhood, lymphocytic lymphoma, cancer of the bladder, cancer of the kidney or ureter, carcinoma of the renal pelvis, neoplasm of the central nervous system (CNS), primary CNS lymphoma, tumour angiogenesis, spinal axis tumour, brain stem glioma, pituitary adenoma, Kaposi's sarcoma, epidermoid cancer, squamous cell cancer, T-cell lymphoma, environmentally induced cancers including those induced by asbestos, and combinations of said cancers.

[41] The composition for use according any one of the preceding items, wherein the composition provides a therapeutically effective dose of the TNFα specific antibody or functional fragment or derivative thereof in the lumen of the ileum or the large intestine of a patient for preventing, minimizing, delaying or alleviating symptoms associated with ICP inhibitor-induced diarrhoea, colitis or enterocolitis.

[42] The composition for use according to any one of the preceding items, wherein the functional antibody fragment or derivative specific to TNFα is a Fab fragment, a F(ab′)₂ fragment, a Fab′ fragment, a scFv, a dsFv, a diabody, a triabody, a tetrabody, an Fc fusion protein, a camelid antibody, a VHH, a vorabody, a VNAR or a minibody.

[43] The composition for use according to any of the preceding items, wherein the antibodies specific to TNFα and functional fragments and derivatives thereof are selected from the group consisting of infliximab, adalimumab, etanercept, certolizumab pegol, golimumab, and functional fragments and derivatives thereof; from anti-TNFα antibodies and functional fragments and derivatives thereof with light chain variable domains and/or heavy chain variable domains comprising complementarity-determining regions (CDRs) with amino acid sequences as disclosed in claim 2 of WO 2017/158092, in claim 2 of, WO 2017/158097, in claim 2 of WO 2017/158079 and/or in claim 2 of WO 2017/158084, as originally filed; from anti-TNFα antibodies and functional fragments and derivatives thereof comprising a heavy chain variable domain amino acid sequence and/or a light chain variable domain amino acid sequence according to claim 4 of WO 2017/158079, claims 5 and 6 of WO 2017/158097, claims 5 and 6 of WO 2017/158092, and claim 4 of WO 2017/158084, as originally filed; and combinations thereof.

[44] The composition for use according to item 43, wherein the anti-TNFα antibody is infliximab, adalimumab, etanercept, certolizumab pegol or golimumab.

[45] The composition for use according to item 43, wherein the anti-TNFα antibody is infliximab.

[46] The composition for use according to item 43, wherein the anti-TNFα antibody is adalimumab.

[47] The composition for use according to any one of items 1 to 43, wherein the functional fragment or derivative is a functional fragment or derivative of infliximab, adalimumab, etanercept, certolizumab pegol or golimumab.

[48] The composition for use according to any of the preceding items, wherein the amino acid sequence of the antibody specific to TNFα or functional fragment or derivative thereof comprises
(i) the amino acids 233P, 234V, 235A, and a deletion at amino acid position 236; and the amino acid 434A or the amino acids 252Y, 254T and 256E; and optionally the amino acids 239D, 330L and 332E or the amino acids 326A, 332E and 333A; and/or
(ii) the amino acids 380A and 434A, and optionally the amino acid 307T; and/or
(iii) the amino acid 434W, and optionally the amino acid 428E and/or the amino acid 311R,
   wherein the amino acid numbering refers to EU numbering.

[49] The composition for use according to any one of items 1 to 43 and 48, wherein the active agent is an antibody comprising a V_{L} domain comprising a CDR1 region having the amino acid sequence as shown in SEQ ID NO:3, a CDR2 region having the amino acid sequence as shown in SEQ ID NO:4, and a CDR3 region having the amino acid sequence as shown in SEQ ID NO:5; and a V_{H} domain comprising a CDR1 region having the amino acid sequence as shown in SEQ ID NO:6, a CDR2 region having the amino acid sequence as shown in SEQ ID NO:7, and a CDR3 region having the amino acid sequence as shown in SEQ ID NO:8.

[50] The composition for use according to any one of items 1 to 43 and 48, wherein the active agent is an antibody comprising a V_{L} domain having the amino acid sequence as shown in SEQ ID NO:10 and a V_{H} domain having the amino acid sequence as shown in SEQ ID NO:9.

[51] The composition for use according to any one of items 1 to 43, 49 and 50 wherein the active agent is an antibody comprising an Fc region comprising amino acids 236 to 451 of the amino acid sequence as shown in SEQ ID NO:11.

[52] The composition for use according to any one of items 1 to 43, wherein the active agent is an antibody comprising a light chain having the amino acid sequence as shown in SEQ ID NO:1 and a heavy chain having the amino acid sequence as shown in SEQ ID NO:11, 14, 15 or 16.

[53] The composition for use according to any one of the preceding items, wherein said treatment comprises the oral administration of the composition.

[54] The composition for use according to item 53, wherein the composition is a solid dosage form in the form of a pellet, granule, micro particle, nano particle, mini tablet, sphere, capsule, tablet or multiparticulate drug delivery system coated with a delayed release coating, which prevents release of the active agent before entering the ileum, the terminal ileum or the ileocolonic region of the gastrointestinal (GI) tract.

[55] The composition for use according to item 54, wherein the delayed release coating comprises at least one component selected from coating materials that disintegrate pH-dependently, coating materials that disintegrate time-dependently, coating materials that disintegrate due to enzymatic triggers in the intestinal environment, and combinations thereof.

[56] The composition for use according to item 53 or 54, wherein
the coating materials that disintegrate pH-dependently are selected from the group consisting of poly vinyl acetate phthalate; cellulose acetate trimellitate; hydroxypropyl methylcellulose phthalate HP-50, HP-55 or HP-55S; cellulose acetate phthalate; hydroxypropyl methylcellulose acetate succinate (HPMCAS); poly(methacrylic acid, ethyl acrylate) 1:1 (Eudragit® L100-55, Eudragit® L30D-55); poly(methacrylic acid, methyl methacrylate) 1:1 (Eudragit® L-100, Eudragit® L12.5); poly(methacrylic acid, methyl methacrylate) 1:2 (Eudragit® S-100, Eudragit® S12,5, Eudragit® FS30D), and combinations thereof;
the coating materials that disintegrate time-dependently are selected from poly(ethyl acrylate, methyl methacrylate) 2:1 (e.g. Eudragit® NM 30D, Eudragit® NE 30D); poly(ethyl acrylate, methyl methacrylate, methacrylic acid 7:3:1 (e.g. Eudragit® RS 30D); ethylcellulose (e.g. Surelease® or Aquacoat ECD); poly(ethyl acrylate, methyl methacrylate, trimethylammonioethyl methacrylate chloride) 1:2:0.2 (e.g. Eudragit® RL 30D); polyvinyl acetate (eg. Kollicoat® SR 30D); and combinations thereof; and
the coating materials that disintegrate due to enzymatic triggers in the intestinal environment are selected from the group consisting of hemicelluloses, chondroitin sulfate; cyclodextrin; pectin; guar gum; chitosan; inulin; lactulose; raffinose; stachyose; alginate; dextran; xanthan gum; locust bean gum; arabinogalactan; amylose; pullulan; carrageenan; scleroglucan; chitin; curdulan; levan; amylopectin; starch; resistant starch; azo compounds being degraded by azo bonds splitting bacteria; and combinations thereof.

[57] The composition according to any one of items 54 to 56, wherein the delayed release coating comprises a combination of at least one coating material that disintegrates pH-dependently and at least one coating material that disintegrates due to enzymatic triggers in the intestinal environment.

[58] The composition according to any one of items 54 to 56, wherein the delayed release coating comprises at least one component selected from the group consisting of hemicelluloses, poly vinyl acetate phthalate, cellulose acetate trimellitate, hydroxypropyl methylcellulose phthalate HP-50, HP-55 or HP-55S, cellulose acetate phthalate, hydroxypropyl methylcellulose acetate succinate (HPMCAS), poly(methacrylic acid, ethyl acrylate) 1:1, poly(methacrylic acid, methyl methacrylate) 1:1, poly(methacrylic acid, methyl methacrylate) 1:2, chondroitin sulfate, pectin, guar gum, chitosan, inulin, lactulose, raffinose, stachyose, alginate, dextran, xanthan gum, locust bean gum, arabinogalactan, amylose, cyclodextrin, pullulan, carrageenan, scleroglucan, chitin, curdulan, levan, amylopectin, starch, resistant starch, azo compounds being degraded by azo bonds splitting bacteria, and combinations thereof.

[59] The composition according to any one of items 53 to 58, wherein, upon oral administration of the composition, the release of the antibody or functional fragment thereof starts in the ileum, the terminal ileum, the ileocolonic region, the ascending colon, transverse colon or the descending colon.

[60] The composition according to any one of items 54 to 59, wherein the delayed release coating comprises a combination of at least one pH sensitive (enteric) polymer, preferably poly(methacrylic acid, methyl methacrylate) 1:2 (e.g. Eudragit® S or L), and at least one polysaccharide selected from hemicelluloses, chondroitin sulfate, cyclodextrin, chitosan, dextran, arabinogalactan, amylose, pullulan, carrageenan, scleroglucan, chitin, curdulan, levan, amylopectin, starch, resistant starch, azo compounds being degraded by azo bonds splitting bacteria, and combinations thereof, preferably resistant starch.

[61] The composition according to any one of items 54 to 59, wherein the delayed release coating comprises, as the sole polymer(s), one or more pH-sensitive polymers, e.g. Eudragit® S and/or L; or wherein the delayed release coating substantially consists of at least one pH-sensitive polymer, e.g. of Eudragit® S and/or L.

[62] The composition for use according to any one of items 1 to 52, wherein said treatment comprises the rectal administration of the composition, and/or wherein the composition is an enema, a gel, a foam or a suppository.

[63] The composition for use according to any of the preceding items, comprising at least one additive selected from hydrophilic polymers, fillers, hydrophilic binders, disintegrants, anti-tacking agents, surfactants, stabilizers, protease resistance enhancers, plasticizers, coalescence agents, lubricants, buffer agents and/or acidifiers.

[64] The composition for use according to any one of the items above, which is an electronic drug capsule engineered to deliver the active agent directly to the ileum and/or colon; or which is provided by genetically modified bacteria expressing TNF-alpha binding proteins.

[65] The composition for use according to any one of the items above, wherein the topical treatment results in a retention of antibody or functional fragment thereof in the gastrointestinal wall, without major systemic release into the rest of the body.

[66] The composition for use according to any one of the preceding items, wherein said treatment comprises administering the antibody or functional fragment thereof once per week, twice per week, once every third day, once every second day, once per day, twice per day, or three times per day to the patient.

[67] The composition for use according to any of the preceding items, in the topical treatment in the ileum, terminal ileum, the cecum, the ascending colon, transverse colon and/or the descending colon of the patient.

[68] The composition for use according to any of the preceding items, wherein the topical treatment of ICP inhibitor-induced diarrhoea, colitis or enterocolitis is indicated by one or more symptoms selected from the group consisting of diarrhoea with less than 4 stools over baseline, diarrhoea with 4-6 stools over baseline, diarrhoea with more than 6 stools over baseline, diarrhoea with more than 7 stools over baseline, abdominal pain, nausea, cramping, blood or mucus in stool or changes in bowel habits, fever, abdominal distention, obstipation and constipation.

[69] The composition for use according to any of the preceding items, wherein the topical treatment prevents, minimizes and/or slows down, or alleviates one or more symptoms associated with ICP inhibitor-induced diarrhoea, colitis or enterocolitis selected from the group consisting of diarrhoea with less than 4 stools over baseline, diarrhoea with 4-6 stools over baseline, diarrhoea with more than 6 stools over baseline, diarrhoea with more than 7 stools over baseline, abdominal pain, nausea, cramping, blood or mucus in stool or changes in bowel habits, fever, abdominal distention, obstipation and constipation.

### BRIEF DESCRIPTION OF THE DRAWINGS

FIG. 1 shows the % body weight change relative to day 0, see Example 3. Data are presented as mean±SE. n=5-12 per group.

FIG. 2 shows the histological score and the endoscopic score of treated animals over time, see Example 3. *: p<0.05, as determined by one-way ANOVA with Dunnett's multiple comparison post-test used to compare all groups to the vehicle control group. Data is presented as mean±SE. n=5-12 per group.

FIG. 3 depicts the histological score at the day of sacrifice (d49), see Example 3. **: p<0.01, ***: p<0.005 as determined by one-way ANOVA with Dunnett's multiple comparison post-test used to compare all groups to the vehicle control group. Data is presented as mean±SE. n=5-12 per group.

FIG. 4 shows the concentrations of cytokines in the colon of treated mice, determined on day 49, see Example 3. *: p<0.05, as determined by one-way ANOVA with Dunnett's multiple comparison post-test used to compare all groups to the vehicle control group. Data is presented as mean±SE. n=5-12 per group.

FIG. 5 shows the effect on histological score following cV1q-huFc treatment, see Example 4. Total histological score which is the sum of the individual scores (0-5 each) for inflammation, crypt damage, erosion, hyperplasia and oedema on day 42 in proximal, mid and distal colon. *: p<0.05, **: p<0.01, ****: p<0.0001 as determined by Kruskal-Wallace with Dunn's multiple comparison post-test used to compare all groups to the respective vehicle control group. Data is presented as mean±SE. n=6-12 per group. Intraperitoneal administration of cV1q was used as control.

FIG. 6 shows the effect on cytokines in colon, see Example 4. Cytokines in colonic tissue were measured by ELISA and results normalized to mg tissue weight. *: p<0.05, **: p<0.01, ***: p<0.005, ****: p<0.001 as determined by one-way ANOVA with Dunnett's multiple comparison post-test used to compare all groups to the vehicle control group. Data is presented as mean±SE. n=6-12 per group. Intraperitoneal administration of cV1q was used as control.

FIG. 7 depicts the mean plasma drug concentration of cV1q-huFc in healthy and colitic Tg32-SCID mice following rectal administration (see Example 5). Plasma samples were collected at 1, 2, 4, 8, 24 and 48 h for single dosed (SD) animals and at 24, 48, 72, 96, 120 and 144 h post first dose for multiple dosed (MD) animals. cV1q-huFc plasma concentrations were determined by Imperacer® Immuno-PCR. The SD and MD profiles are based on two subgroups due to limitations in blood collection (SD: 1, 4 and 24h from subgroup 1 and 2, 8 and 48 h from subgroup 2; MD: 24, 72 and 120 h from subgroup 1 and 48, 96 and 144 h from subgroup 2). Data are shown as mean±SD (n=5-7).

FIG. 8 shows cV1q-huFc median plasma concentrations following intracaecal administration to healthy and colitic mice (see Example 6). Plasma samples were collected at sacrifice at 1, 4, 8, 12 and 24 h after administration of a single dose to Tg32-SCID mice and at 120 h post first dose for multiple dosed animals. cV1q-huFc plasma concentrations were determined by Imperacer® Immuno-PCR. Data are shown as median±IQR (n=4). IC: intracaecal; IR: intra-rectal.

FIG. 9 shows cV1q-huFc median concentrations in proximal and distal colon following intracaecal or rectal administration (see Example 6). Proximal and distal colon tissue samples were collected at sacrifice at 1, 4, 8, 12 and 24 h after administration of a single dose to Tg32-SCID mice and at 120 h post first dose for multiple dosed animals. Tissue samples were rinsed, weighed and homogenized in 0.3 mL PBS. cV1q-huFc concentrations in supernatant were determined using a commercially available IgG ELISA. Obtained concentrations (pg/mL) were then normalized to tissue weight. Data are shown as median±IQR (n=4). IC: intracaecal; IR: intra-rectal. *: Median value was BLQ in the proximal colon at 1 and 4 h for healthy animals, at 1, 4, and 8h for colitic mice treated with 300 μg cV1q-huFc and in the distal colon at 12 h for colitic mice treated with 100 μg.

FIG. 10 shows the effect of Ab-REW and infliximab on IFNγ production after nivolumab treatment (see Example 7). CD14⁺ monocytes were isolated from PBMC and differentiated over a period of 7 days into monocyte-derived dendritic cells (mo-DC) by the addition of GM-CSF and IL-4 to the cell culture medium. CD4⁺ T-cells were also isolated from PBMC. CD4⁺ T-cells and mo-DC were combined at a T-cell:DC ratio of 5:1 and incubated together with nivolumab (10 μg/mL) and serially diluted Ab-REW or infliximab (50-0.003 μg/mL) for 5 days. IFNγ concentrations in the cell culture supernatants were then determined by ELISA. Data are represented as mean (±SE) with n=3 technical replicates. 4-PL non-linear regression was performed where possible. The lower dotted line represents the average secretion of IFNγ from untreated cells. The upper dotted line represents the average secretion of IFNγ in the presence of nivolumab only (n=6 for all controls).

### DETAILED DESCRIPTION OF THE INVENTION

The present invention is described in the following with regard to antibodies specific to tumour necrosis factor alpha (TNFα) and functional fragments and derivatives thereof, which represent the most preferred embodiments. All embodiments described hereinafter equally apply to antibodies and functional fragments and derivatives thereof directed to other targets (antigens) mutatis mutandis. The targets of these antibodies and functional fragments thereof include, but are not limited to, anti-inflammatory cytokines such as IL-13 as well as their receptors; pro-inflammatory cytokines, such as CD20, IL-6, IL-12 and IL-23 (IL-12/IL-23p40, IL-23p19), IL-17, IL-21 as well as their receptors; cell adhesion molecules, such as MadCAM-1, ICAM-1; C-C chemokine receptors, such as CCR5, CCR9 and their ligands; integrins, such as alpha4beta7, beta7, alpha2beta1, alphaEbeta7; toll-like receptors, such as TLR2, TLR9; eotaxins, such as Eotaxin-1; members of the tumour necrosis factor receptor superfamily, such as OX40; matrix metalloproteinases, such as MMP-9; C—X-C motif chemokines, such as IP-10; and other proteins, such as CD20. Preferred targets are α4β7 integrin and IL-23.

The present invention, according to a first aspect of the invention, relates to a pharmaceutical composition comprising an active agent selected from the group consisting of antibodies specific to tumour necrosis factor alpha (TNFα) and functional fragments and derivatives thereof, for use in the treatment or prevention of at least one adverse event of the gastro-intestinal tract induced by a cancer therapy in a patient, preferably induced by one or more immune checkpoint (ICP) inhibitors, wherein said treatment or prevention comprises the topical administration of said composition to the ileum and/or the large intestine of said patient.

The invention further relates to a pharmaceutical composition comprising an active agent selected from the group consisting of antibodies specific to tumour necrosis factor alpha (TNFα) and functional fragments and derivatives thereof, for use in the topical treatment in the ileum and/or the large intestine of a patient suffering from immune checkpoint (ICP) inhibitor-induced colitis or enterocolitis or diarrhoea. This inventive composition allows for uninterrupted systemic treatment with one or more ICP inhibitors. This has the advantage that the successful cancer treatment with the ICP inhibitor does not have to be compromised. Moreover, the topical treatment with antibodies specific to TNFα and functional fragments and derivatives thereof means that the use of corticosteroids as well as of systemically applied antibodies specific to TNFα and functional fragments and derivatives thereof is not required, thereby minimizing undesirable side-effects of such treatments.

In the context of the present invention, the term “antibody” is used as a synonym for “immunoglobulin” (Ig), which is defined as a protein belonging to the class IgG, IgM, IgE, IgA, or IgD (or any subclass thereof), and includes all conventionally known antibodies and functional fragments and derivatives thereof. In the context of the present invention, a “functional fragment or derivative” of an antibody/immunoglobulin is defined as antigen-binding fragment or other derivative of a parental antibody that essentially maintains the antigen-binding properties of such a parental antibody. In a preferred embodiment, the functional fragment or derivative of the antibody comprises a functional Fc portion exhibiting at least effector function. In one embodiment, the antibody is capable of binding to human FcRn.

An “antigen-binding fragment or derivative” of an antibody/immunoglobulin is defined as a fragment (e.g., a variable region of an IgG) or derivative that retains the antigen-binding region.

An “antigen-binding region” of an antibody typically is found in one or more hypervariable region(s) of an antibody, i.e., the CDR-1, -2, and/or -3 regions. “Antigen-binding fragments” of the invention include the domain of a F(ab′)₂ fragment and a Fab fragment. “Functional fragments and derivatives” of the invention include Fab fragment, F(ab′)2 fragment, Fab′ fragment, scFv, dsFv, diabody, triabody, tetrabody, Fc fusion protein, vorabody, camelid antibody, VHH, VNAR and minibody. The F(ab′)2 or Fab domain may be engineered to minimize or completely remove the intermolecular disulphide interactions that occur between the CH1 and CL domains. The antibodies or functional fragments of the present invention may be part of bi- or multifunctional constructs.

Functional fragments and derivatives of the present invention include, but are not limited to, Fab fragments, F(ab′)₂ fragments, Fab′ fragments, scFv and diabodies.

Fab fragments can be obtained as the purified digestion products after digestion of a TNFα-specific antibody with a cysteine proteinase like papain (EC 3.4.22.2). F(ab′)₂ fragments can be obtained as the purified digestion products after digestion of a TNFα-specific antibody with pepsin (EC 3.4.23.1) or IdeS (Immunoglobulin degrading enzyme from Streptococcus pyogenes; EC 3.4.22). Fab′ fragments can be obtained from F(ab′)₂ fragments in mild reducing conditions, whereby each F(ab′)₂ molecule gives rise to two Fab′ fragments.

A scFv is a single chain Fv fragment in which the variable light (“V_{L}”) and variable heavy (“V_{H}”) domains are linked by a peptide bridge.

A “diabody” is a dimer consisting of two fragments, each having variable regions joined together via a linker or the like (hereinafter referred to as diabody-forming fragments), and typically contain two Vis and two V_{H}s. Diabody-forming fragments include those consisting of V_{L} and V_{H}, V_{L} and V_{L}, V_{H} and V_{H}, etc., preferably V_{H} and V_{L}. In diabody-forming fragments, the linker joining variable regions is not specifically limited, but preferably short enough to avoid noncovalent bonds between variable regions in the same fragment. The length of such a linker can be determined as appropriate by those skilled in the art, but typically 2-14 amino acids, preferably 3-9 amino acids, especially 4-6 amino acids are used. In this case, the V_{L} and V_{H} encoded on the same fragment are joined via a linker short enough to avoid noncovalent bonds between the V_{L} and V_{H} on the same chain and to avoid the formation of single-chain variable region fragments so that dimers with another fragment can be formed. The dimers can be formed via either covalent or noncovalent bonds or both between diabody-forming fragments.

Moreover, diabody-forming fragments can be joined via a linker or the like to form single-chain diabodies (sc(Fv)₂). By joining diabody-forming fragments using a long linker of about 15-20 amino acids, noncovalent bonds can be formed between diabody-forming fragments existing on the same chain to form dimers. Based on the same principle as for preparing diabodies, polymerized antibodies such as trimers or tetramers can also be prepared by joining three or more diabody-forming fragments.

In one embodiment, the functional antibody fragment or derivative is a Fab fragment, a F(ab′)2 fragment, a Fab′ fragment, an scFv, a dsFv, a diabody, a triabody, a tetrabody, an Fc fusion protein, a vorabody, camelid antibody, a VHH, a VNAR or a minibody. Preferred functional fragments or derivatives used in the present invention are Fab fragments, F(ab′)₂ fragments, Fab′ fragments, scFv and diabodies.

The antibody or functional fragment or derivative thereof comprised in the composition of the invention specifically binds to TNFα, but is otherwise not particularly limited. The terms “anti-TNFα antibody”, “TNFα antibody”, “TNFα specific antibody” and “antibody specific to TNFα” as used herein are interchangeable. In its most general form (and when no defined reference is mentioned), “specific” and “specific binding” to the ability of the antibody or functional fragment or derivative to discriminate between human TNFα and an unrelated biomolecule, as determined, for example, in accordance with a specificity assay methods known in the art. Such methods comprise, but are not limited to, Western blots and enzyme-linked immunosorbent assay (ELISA) tests. For example, a standard ELISA assay can be carried out. Typically, determination of binding specificity is performed by using not a single reference biomolecule, but a set of about three to five unrelated biomolecules, such as milk powder, BSA, transferrin or the like. In one embodiment, specific binding refers to the ability of the antibody or fragment to discriminate between human TNFα and human TNFβ. In a preferred embodiment of the present invention the TNFα antibody or functional fragment thereof is a TNFα antibody. In an alternatively preferred embodiment of the present invention the TNFα antibody or functional fragment thereof is a functional fragment or derivative of a TNFα antibody.

In a preferred aspect of the invention, the antibody of the invention is a non-fucosylated antibody or an antibody having reduced fucosylation. The term “antibody having reduced fucosylation”, as used herein, refers to an antibody in which less than 90% of the N-glycans of the antibody are fucosylated. Methods to determine the percentage of fucosylation are known in the art. In one embodiment, less than 75%, or less than 50%, or less than 25% of the N-glycans of the antibody are fucosylated. Most preferably, less than 10% of the N-glycans of the antibody are fucosylated. In a particular embodiment, the N-glycans of the antibody of the invention do not contain any fucose. Preferably, less than 90% of the N-glycans at N297 (EU numbering) of the antibody are fucosylated. In another embodiment, less than 75%, or less than 50%, or less than 25% of the N-glycans at N297 (EU numbering) of the antibody are fucosylated. Most preferably, less than 10% of the N-glycans at N297 (EU numbering) of the antibody are fucosylated. In another embodiment, the N-glycans at N297 of the antibody do not contain any fucose.

Non-fucosylated antibodies, sometimes also referred to as afucosylated antibodies, can be generated by various methods. For example, the synergistic knockdown of the genes for α1,6-fucosyltransferase (FUT8) and GDP-mannose 4,6-dehydratase (GMD) in CHO cells can be used to produce monoclonal antibody variants that are fully afucosylated and ADCC-enhanced (see, e.g., Imai-Nishiya et al. (2007) BMC Biotechnol. 7, 84). A method using zinc-finger nucleases (ZFNs) cleaving the FUT8 gene in a region encoding the catalytic core of the α1,6-fucosyltransferase and thus disrupting the corresponding enzymatic function in CHO cells can be used to produce monoclonal antibodies completely lacking core fucose (see, e.g., Malphettes et al. (2010) Biotechnol. Bioeng. 106, 774-783).

Antibodies having reduced fucosylation can be prepared by addition of a decoy substrate such as 2-deoxy-2-fluoro-2-fucose to the culture medium (see, e.g., Dekker et al. (2016) Sci Rep 6:36964), resulting in a reduced incorporation of fucose in the IgG-Fc glycans.

In another embodiment, the antibody of the invention has a high sialic acid content. An increase in sialylation can be achieved, e.g. by simultaneous transfection of cytidine monophosphate-sialic acid synthase (CMP-SAS), cytidine monophosphate-sialic acid transporter (CMP-SAT), and a 2,3-sialyltransferases (see, e.g., Son et al. (2011) Glycobiology 21, 1019-1028).

Preferably, the affinity at pH 6 to human FcRn of the antibody of the invention is high. The high affinity binding of the antibody to human FcRn at pH 6 is characterized by a K_{D} value of less than 500 nM. Preferably, the K_{D} value of the high affinity binding at pH 6 is less than 400 nM, or less than 300 nM, or less than 200 nM. For example, the K_{D} value characterizing the affinity at pH 6 may be in the range from 1 to 500 nM, or 2 to 400 nM, or 3 to 300 nM, or 4 to 200 nM, or 5 to 100 nM.

In one embodiment, the affinity of the antibody of the invention to human FcRn at pH 6 is greater than the affinity of infliximab to human FcRn at pH 6.0.

The affinity of the antibody of the invention to human FcRn is preferably determined by surface plasma resonance (SPR).

Several monoclonal antibodies against TNFα have been described in the prior art. Meager et al. (Hybridoma, 6, 305-311, 1987) describe murine monoclonal antibodies against recombinant TNFα. Fendly et al. (Hybridoma, 6, 359-370, 1987) describe the use of murine monoclonal antibodies against recombinant TNFα in defining neutralising epitopes on TNF. Furthermore, in international patent application WO 92/11383, recombinant antibodies, including CDR-grafted antibodies, specific for TNFα are disclosed. U.S. Pat. No. 5,919,452 discloses anti-TNFα chimeric antibodies and their use in treating pathologies associated with the presence of TNFα. Further anti-TNFα antibodies are disclosed in Stephens et al. (Immunology, 85, 668-674, 1995), GB-A-2 246 570, GB-A-2 297 145, U.S. Pat. No. 8,673,310, US 2014/0193400, EP 2 390 267 B1, U.S. Pat. Nos. 8,293,235, 8,697,074, WO 2009/155723 A2 and WO 2006/131013 A2.

Currently approved anti-TNFα antibodies include (i) infliximab, a chimeric IgG monoclonal antibody (Remicade®, Inflectra®, Remsima®); (ii) etanercept, a TNFR2 dimeric fusion protein, with an IgG1 Fc (Enbrel®); (iii) adalimumab, a fully human monoclonal antibody (mAb) (Humira®), (iv) certolizumab pegol, a PEGylated Fab fragment (Cimzia®) and (v) golimumab, a human IgG1 monoclonal antibody (Simponi®). Any reference herein to an INN of an antibody or functional fragment or derivative thereof encompasses biosimilar and biobetter versions thereof. In one embodiment of the present invention, the anti-TNFα antibody or functional fragment or derivative thereof is selected from infliximab, adalimumab, etanercept, certolizumab pegol and golimumab or functional fragments or derivatives thereof. In another embodiment of the present invention, the antibody or functional fragment or derivative thereof is selected from anti-TNFα antibodies or functional fragments or derivatives thereof as disclosed in WO 2017/158092, WO 2017/158097, WO 2017/158084 and WO 2017/158079. In yet another embodiment of the present invention, the at least one antibody or functional fragment or derivative thereof is an anti-TNFα antibody or functional fragment or derivative thereof with a light chain variable domain and/or a heavy chain variable domain comprising complementarity-determining regions (CDRs) with amino acid sequences as disclosed in PCT applications WO 2017/158092, WO 2017/158097, WO 2017/158084 and WO 2017/158079.

In a preferred embodiment of the present invention, the antibody or functional fragment or derivative thereof is selected from anti-TNFα antibodies or functional fragments or derivatives thereof with a light chain variable domain and/or a heavy chain variable domain comprising one or more CDRs with amino acid sequences as disclosed in SEQ ID NO:7, 9, 12, 14, 24 and 25 of WO 2017/158079, in SEQ ID NO:7-11 and 6 of WO 2017/158097, in SEQ ID NO:7-12 of WO 2017/158092, in SEQ ID NO:1-4, 7 and 6 of WO 2017/158084, and combinations thereof. In another preferred embodiment of the present invention, the antibody or functional fragment or derivative thereof is selected from anti-TNFα antibodies or functional fragments or derivatives thereof with a light chain variable domain and a heavy chain variable domain comprising CDRs with amino acid sequences as disclosed in claim 2 of WO 2017/158079, in claim 2 of WO 2017/158097, in claim 2 of WO 2017/158092 and/or in claim 2 of WO 2017/158084, as originally filed. In yet another preferred embodiment of the present invention, the anti-TNFα antibody or functional fragment or derivatives thereof is selected from the group consisting of anti-TNFα antibodies or functional fragments or derivatives thereof comprising a heavy chain variable domain amino acid sequence and/or a light chain variable domain amino acid sequence according to claim 4 of WO 2017/158079, claims 5 and 6 of WO 2017/158097, claims 5 and 6 of WO 2017/158092, claim 4 of WO 2017/158084, and combinations thereof.

In a preferred embodiment the antibody comprises (i) a V_{L} domain comprising a CDR1 region having the amino acid sequence as shown in SEQ ID NO:3, a CDR2 region having the amino acid sequence as shown in SEQ ID NO:4, and a CDR3 region having the amino acid sequence as shown in SEQ ID NO:5; and (ii) a V_{H} domain comprising a CDR1 region having the amino acid sequence as shown in SEQ ID NO:6, a CDR2 region having the amino acid sequence as shown in SEQ ID NO:7, and a CDR3 region having the amino acid sequence as shown in SEQ ID NO:8. More preferably the antibody comprises a V_{L} domain having the amino acid sequence as shown in SEQ ID NO:10 and a V_{H} domain having the amino acid sequence as shown in SEQ ID NO:9.

In another preferred embodiment (which can be combined with any one of the embodiments in the preceding paragraph) the antibody comprises an Fc region comprising amino acids 236 to 451 of the amino acid sequence as shown in SEQ ID NO:11.

Most preferably the antibody comprises a light chain having the amino acid sequence as shown in SEQ ID NO:1 and a heavy chain having the amino acid sequence as shown in SEQ ID NO:11, 14, 15 or 16.

In one embodiment the antibody comprises a light chain having the amino acid sequence as shown in SEQ ID NO:1 and a heavy chain having the amino acid sequence as shown in SEQ ID NO:11. In another embodiment, the antibody comprises a light chain having the amino acid sequence as shown in SEQ ID NO:1 and a heavy chain having the amino acid sequence as shown in SEQ ID NO:14. In another embodiment, the antibody comprises a light chain having the amino acid sequence as shown in SEQ ID NO:1 and a heavy chain having the amino acid sequence as shown in SEQ ID NO:15. In another embodiment, the antibody comprises a light chain having the amino acid sequence as shown in SEQ ID NO:1 and a heavy chain having the amino acid sequence as shown in SEQ ID NO:16.

In yet another embodiment, the antibody comprises a light chain consisting of the amino acid sequence as shown in SEQ ID NO:1 and a heavy chain consisting of the amino acid sequence as shown in SEQ ID NO:11. In yet another embodiment, the antibody comprises a light chain consisting of the amino acid sequence as shown in SEQ ID NO:1 and a heavy chain consisting of the amino acid sequence as shown in SEQ ID NO:14. In yet another embodiment, the antibody comprises a light chain consisting of the amino acid sequence as shown in SEQ ID NO:1 and a heavy chain consisting of the amino acid sequence as shown in SEQ ID NO:15. In yet another embodiment, the antibody comprises a light chain consisting of the amino acid sequence as shown in SEQ ID NO:1 and a heavy chain consisting of the amino acid sequence as shown in SEQ ID NO:16.

In a further embodiment, the TNFα antibody is infliximab, adalimumab, etanercept, certolizumab pegol or golimumab. In an alternative further embodiment, the functional fragment or derivative of the TNFα antibody is a functional fragment or derivative of infliximab, adalimumab, etanercept, certolizumab pegol or golimumab.

The antibody specific to TNFα or functional fragment or derivative thereof may further comprise one or more modifications, e.g. in the form of added or substituted residues, that improve stability, specificity or targeting. These may include any such modifications that are known in the art. In one embodiment of the present invention, the at least one antibody specific to TNFα or functional fragment or derivative thereof comprises one or more modifications of any such modifications disclosed in U.S. Pat. Nos. 8,871,204, 6,737,056, 8,742,074, WO 2002/060919, WO 2017/158426, international patent applications PCT/EP2018/074525 (WO 2019/057567 A1), PCT/E P2018/074523 (WO 2019/057565 A1), or PCT/EP2018/074522 (WO 2019/057564 A1).

In a specific embodiment, the amino acid sequence of the antibody heavy chain comprises i) the amino acids 233P, 234V, 235A, and a deletion at amino acid position 236; and the amino acid 434A or the amino acids 252Y, 254T and 256E; and optionally the amino acids 239D, 330L and 332E or the amino acids 326A, 332E and 333A (EU numbering); and/or ii) the amino acids 380A and 434A, and optionally the amino acid 307T (EU numbering); and/or iii) the amino acid 434W, and optionally the amino acid 428E and/or the amino acid 311R (EU numbering). In another specific embodiment, the amino acid sequence of the antibody heavy chain comprises mutations at one or more of positions selected from the group consisting of 311, 428, 434, 435, and 438 (EU numbering). Preferably, the amino acid sequence of the antibody heavy chain comprises the amino acid 434W, the amino acid 428E and the amino acid 311R (EU numbering). Most preferably, the antibody comprises an Fc domain comprising amino acids 236 to 451 of the amino acid sequence as shown in SEQ ID NO:11. Antibodies having the specific amino acids described in this paragraph may be obtained by introducing substitutions into a heavy chain sequence, e.g. into the sequence as shown in SEQ ID NO:2.

Unless stated otherwise herein, references to residue numbers in the amino acid sequence of antibodies corresponds to the residue numbering by the EU numbering system, also called the EU index, as described in Kabat et al., Sequences of Proteins of Immunological Interest, 5th Ed. Public Health Service, National Institutes of Health, Bethesda, Md., 1991. (see e.g. WO 2006/073941).

The inventive pharmaceutical composition may comprise as an active agent one type of antibody specific to TNFα or functional fragments or derivatives thereof, or several different antibodies specific to TNFα and/or functional fragments or derivatives of antibodies specific to TNFα. For example, the inventive composition may comprise 1, 2, 3, 4, or 5 different antibodies specific to TNFα and functional fragments or derivatives of antibody specific of TNFα.

As used herein, the terms “treat,” “treating,” and “treatment” with regard to ICP inhibitor-induced diarrhoea, colitis or enterocolitis relate to the administration of TNFα specific antibodies or functional fragments or derivatives thereof, as part of the inventive composition, to prevent, minimize or delay the onset of the symptoms, complications, or similar biochemical indicia of ICP inhibitor-induced diarrhoea, colitis or enterocolitis, or to alleviate or minimize symptoms, complications, or similar biochemical indicia of ICP inhibitor-induced diarrhoea, colitis or enterocolitis.

The term “topical treatment” in the context of the present invention, is used to describe the local application of the composition, as opposed to the systemic application of TNFα antibody containing compositions, e.g. by intravenous infusion or subcutaneous injection, used in commercial products. However, the topical treatment in the lumen of the ileum and large intestine is not limited by the way of administration of the composition. The term “administration” in this context relates to the manner and form in which the composition comes into first contact with the body of a patient. This means that the composition in a suitable form can be administered orally, rectally or in any other way that results in the accumulation of the composition at the site of local application.

In the present invention the term “topical treatment in the ileum and/or the large intestine” refers to the local application, as defined above, in the lumen of the ileum and/or the large intestine, in other words the local application somewhere in the combined and continuous inside made up by the ileum of the small intestine and the large intestine. The “large intestine” is the last section of the gastrointestinal (GI) tract and can be further subdivided into caecum, colon and rectum. The “colon” can be further subdivided into ascending, transverse and descending colon. The “ileum” of the small intestine is the last section of the small intestine and is at one end connected to the caecum. The “terminal ileum” is the last section of the ileum, which is directly adjacent to the caecum. The term “gastrointestinal tract” or “GI” as used herein describes the system of organs of the human body, that includes all structures between mouth and anus, forming a continuous passage, and is responsible for digesting ingested material, absorbing nutrients and expelling faeces. In one embodiment of the present invention the inventive composition is for use in the topical treatment in the terminal ileum, and/or the large intestine, preferably the colon. In another embodiment of the present invention the inventive composition is for use in the topical treatment in in the terminal ileum, the caecum, the ascending colon, transverse colon and/or the descending colon of the patient.

The topical treatment with the pharmaceutical composition of the present invention allows the local application of the TNFα antibodies and functional fragments or derivatives thereof in the lumen of the ileum and/or the large intestine of a patient. Thereby, systemic application of the TNFα antibodies and functional fragments or derivatives thereof can be avoided. In this way systemic uptake and distribution of TNFα antibodies and functional fragments or derivatives thereof can be minimized. Thus, the TNFα antibodies and functional fragments or derivatives thereof do not significantly counteract the successful cancer treatment with ICP inhibitors on a systemic level. Moreover, the multitude of risks associated with systemic inhibition of the immune defence function of TNFα in a patient, including for example infectious complications and build-up of antibodies specific to anti-TNFα antibody in the body of the patient resulting in a loss of response to anti-TNFα antibodies can be kept to a minimum.

The inventive composition, according to the first aspect of the invention, is for use in the treatment of a patient suffering from ICP inhibitor-induced adverse events in the ileum and/or the colon, namely ICP inhibitor-induced diarrhoea, colitis or enterocolitis.

As used herein, an “ICP inhibitor” is any molecule (e.g. small molecule, protein, peptide, nucleic acid molecule, or antibody) that is administered to a patient to stimulate the patient's immune system (e.g. by unblocking an existing immune response or by unblocking the initiation of an immune response) for the purpose of treating a disease (e.g. a cancer or an infectious disease). ICP inhibitors may target any component immune checkpoint known in the art that results in the stimulation of the immune system, including but not limited to, CTLA-4, PD-1, PD-L1, PDL2, B7-H3, B7-H4, BTLA, HVEM, TIM3, GAL9, LAG3, CSF-1R, VISTA, KIR, 2B4, CD160, CGEN-15049, CHK1, CHK2, A2aR, ADAR1, CD47 (targeted by CD47 inhibitors), ICOS (targeted by ICOS agonists), TIGIT (targeted by TIGIT inhibitors) and the B-7 family of ligands. Combinations of inhibitors for a single component of the immune checkpoint or different inhibitors for different components of the immune checkpoint may be used. Immune checkpoints are typically part of, or consist of, inhibitory or stimulatory pathways that maintain self-tolerance and assist with immune response. ICP inhibitors in accordance with the present invention include molecules blocking a pathway and molecules stimulating a pathway. For example, TIGIT inhibitors and ICOS agonists are ICP inhibitors in the sense of the present invention. Marin-Acevedo et al. (2018) Journal of Hematology & Oncology 11, Article number 39 summarizes inhibitory and stimulatory pathways and their targets that can be targeted by suitable ICP inhibitors. The content of Marin-Acevedo et al. is incorporated herein in its entirety.

An “adverse event” as used herein is any unfavourable and generally unintended, even undesirable, sign (including an abnormal laboratory finding), symptom, or disease associated with the use of a medical treatment. For example, an adverse event may be associated with activation of the immune system or expansion of immune system cells (e.g., T-cells) in response to a treatment. A medical treatment may have one or more associated adverse events and each adverse event may have the same or different level of severity.

As used herein in the context of the present invention, “colitis” refers to an inflammatory condition of the large intestine, and in particular the colon, that can be associated with symptoms including diarrhoea, abdominal pain, nausea, cramping, blood or mucus in stool or changes in bowel habits, fever, abdominal distention, obstipation, and oedematous, hyperaemic, and/or friable wall of the large intestine. As used herein in the context of the present invention, “enterocolitis” refers to an inflammatory condition of the large intestine, and in particular the colon, and the ileum, preferably the terminal ileum, of the small intestine that can be associated with symptoms including diarrhoea, abdominal pain, nausea, cramping, blood or mucus in stool or changes in bowel habits, fever, abdominal distention, obstipation and constipation, or oedematous, hyperaemic, and/or friable wall of the ileum or the large intestine.

As used herein, the terms “ICP inhibitor-induced colitis” and “ICP inhibitor-induced enterocolitis” refer to a colitis and an enterocolitis, respectively, that: (1) have their first occurrence in a patient concurrent with, or shortly after (i.e., days or weeks, for example 1 to 60 weeks, 1 to 48 weeks, 13 to 60 weeks, or 7 to 26 weeks after), first administration of one or more ICP inhibitors; (2) are identified as an ICP inhibitor-induced colitis or enterocolitis, respectively, by a physician, e.g. based on criteria set out in the ASCO Practice Guideline; and (3) are not identified as a colitis or enterocolitis, respectively, of another aetiology (e.g., Clostridium difficile toxin) by a physician.

Except when specifically noted, the terms “patient” or “subject” are used interchangeably and refer to mammals such as human patients and non-human primates, as well as experimental animals such as rabbits, rats, and mice, and other animals. Animals include all vertebrates, e.g. mammals and non-mammals, such as sheep, dogs, cows, chickens, amphibians, and reptiles.

ICP inhibitor-induced colitis may be graded for a living patient into grades 1 to 4, based on the toxicities occurring in the large intestine, and in particular in the colon, as a consequence of the treatment of a patient with one or more ICP inhibitors. The grading for ICP inhibitor-induced colitis is as follows:
Grade 1 colitis presents as an asymptomatic with only radiographic or histologic findings.
Grade 2 colitis is characterized by the presence of mucus or blood in stool and abdominal pain;
Grade 3 colitis is characterized by severe abdominal pain, fever, change in bowel habits and peritoneal signs.
Grade 4 colitis is a life-threatening condition with signs of perforation or bleeding or ischaemia or necrosis and development of toxic megacolon.
Grade 5 colitis is death caused by colitis

Similarly, ICP inhibitor-induced enterocolitis, may be graded into grade 1 to 4 based on the toxicities occurring in the small intestine, and in particular in the ileum, and the large intestine, as a consequence of the treatment of a patient with one or more ICP inhibitors, and in particular in the colon. The grading for ICP inhibitor-induced enterocolitis is as follows:
Grade 1 enterocolitis presents as an asymptomatic with only radiographic or histologic findings.
Grade 2 enterocolitis is characterized by the presence of mucus or blood in stool and abdominal pain;
Grade 3 enterocolitis is characterized by severe or persistent abdominal pain, fever, ileus and peritoneal signs.
Grade 4 enterocolitis is a life-threatening condition with signs of perforation or bleeding or ischaemia or necrosis and development of toxic megacolon.
Grade 5 enterocolitis is death caused by enterocolitis

ICP-inhibitor-induced diarrhoea is graded as follows:
Grade 1 diarrhoea is defined as an increase of <4 stools over baseline;
Grade 2 diarrhoea is characterized by 4-6 stools over baseline;
Grade 3 diarrhoea is characterized by 7 stools over baseline;
Grade 4 diarrhoea is characterized by life threatening consequences due to the diarrhoea;
Grade 5 diarrhoea is death.

The diagnostic work-up to determine the toxicities for grading the ICP inhibitor-induced diarrhoea, colitis or enterocolitis can be carried for example as described in the ASCO Practice Guideline (Brahmer et al., Management of Immune-Related Adverse Events in Patients Treated With Immune Checkpoint Inhibitor Therapy: American Society of Clinical Oncology Practice Guideline, J Clin Oncol. 2018 Jun. 10; 36(17):1714-1768). According to the ASCO Practice Guideline, diagnostic work-up for grade 2 toxicities should include the following:
work-up of blood (CBC, comprehensive metabolic panel, thyroid-stimulating hormone [TSH], erythrocyte sedimentation rate [ESR], C-reactive protein [CRP]), stool (culture, Clostridium difficile, parasite, cytomegalovirus [CMV] or other viral aetiology, ova and parasite) should be performed;
may test for lactoferrin for patient stratification to determine who needs urgent endoscopy, and calprotectin may be offered to follow up on disease activity;
screening laboratories (HIV, hepatitis A and B, and blood quantiferon for TB) to prepare patients to start infliximab should be routinely done in patients at high risk for those infections and in appropriately selected patients based on infectious disease expert's evaluation;
imaging with computed tomography (CT) scan of abdomen and pelvis and GI endoscopy with biopsy may be performed as there is evidence showing that the presence of ulceration in the colon can predict a corticosteroid-refractory course, which may require early infliximab. Infliximab or other tumour necrosis factor (TNF)-blocking agent should not be delayed while awaiting the results of these screening tests;
repeat endoscopy may be offered to patients who do not respond to immunosuppressive agents. Repeating endoscopy for disease monitoring should only be offered when clinically indicated and when planning to resume therapy.

According to the ASCO Practice Guideline, diagnostic work-up for grade 3 to 4 toxicities should include the following:
All the work-up listed for Grade 2 (blood, stool, imaging, and scope with biopsy) should be completed immediately;
repeat endoscopy may be offered for patients who do not respond to immunosuppressive agents. Repeating endoscopy for disease monitoring should only be offered when clinically indicated and when planning to resume ICP inhibitor.

According to one embodiment of the invention, the treatment with the antibody or fragment or derivative thereof prevents the progression of the adverse effect (e.g. diarrhoea, colitis and/or enterocolitis) to a higher grade toxicity. The following embodiments are preferred.

The patient to be treated suffers from an adverse effect (e.g. diarrhoea, colitis and/or enterocolitis) of grade 1 toxicity, and the treatment with the antibody or fragment or derivative thereof prevents progression of the adverse effect to a higher grade toxicity, e.g. to grade 2 toxicity.

The patient to be treated suffers from an adverse effect (e.g. diarrhoea, colitis and/or enterocolitis) of grade 2 toxicity, and the treatment with the antibody or fragment or derivative thereof prevents progression of the adverse effect to a higher grade toxicity, e.g. to grade 3 toxicity.

The patient to be treated suffers from an adverse effect (e.g. diarrhoea, colitis and/or enterocolitis) of grade 3 toxicity, and the treatment with the antibody or fragment or derivative thereof prevents progression of the adverse effect to a higher grade toxicity, e.g. to grade 4 toxicity.

In one aspect the invention relates to an active agent selected from the group consisting of antibodies specific to tumour necrosis factor alpha (TNFα) and functional fragments and derivatives thereof, for use in preventing the progression or worsening of at least one adverse event of the gastro-intestinal tract induced by a cancer therapy in a patient, preferably induced by one or more immune checkpoint (ICP) inhibitors, wherein said prevention comprises the topical administration of said composition to the ileum and/or the large intestine of said patient. Preferably, the topical administration is oral administration. Preferably the adverse event is selected from the group consisting of diarrhoea, colitis, enterocolitis and combinations thereof, and the progression from grade 1 toxicity to grade 2 toxicity or any higher grade is prevented. In another embodiment the adverse event is selected from the group consisting of diarrhoea, colitis, enterocolitis and combinations thereof, and the progression from grade 2 toxicity to grade 3 toxicity or any higher grade is prevented. In another embodiment the adverse event is selected from the group consisting of diarrhoea, colitis, enterocolitis and combinations thereof, and the progression from grade 3 toxicity to grade 4 toxicity is prevented. The preferred embodiments of this aspect of the invention correspond to the preferred embodiments of the other aspects of the invention described herein mutatis mutandis.

According to another embodiment of the invention, the patient to be treated with the antibody or fragment or derivative thereof is suffering from ICP inhibitor-induced diarrhoea but not of colitis or enterocolitis, and said treatment prevents the development or onset of colitis and enterocolitis in said patient.

According to one embodiment of the first aspect of the present invention the patient is also undergoing treatment with one or more ICP inhibitors. “Also undergoing treatment with one or more ICP inhibitors” means that the treatment has not been finally discontinued, but is presently ongoing or has been interrupted temporarily. According to a preferred embodiment the patient is also undergoing systemic treatment with one or more ICP inhibitors. Systemic treatment or therapy refers to a treatment reaching and affecting substantially all cells of the body via the systemic circulation.

Before administration of the composition of the present invention, the treatment with one or more ICP inhibitors, upon development of toxicities associated with ICP inhibitor-induced diarrhoea, colitis or enterocolitis may be temporarily interrupted for weeks, or days, or not at all. According to one embodiment of the present invention, treatment with one or more ICP inhibitors is interrupted for less than four weeks, preferably less than two weeks, more preferably one week, even more preferably less than five, four, three, two days or one day, and most preferably wherein treatment is not interrupted.

The toxicity grade of the patient with ICP inhibitor-induced diarrhoea, colitis or enterocolitis, to which the composition of the present invention is to be administered is not particularly limited. The toxicity grade of the ICP inhibitor-induced diarrhoea, colitis or enterocolitis may be determined by a physician, e.g. applying the ASCO Practice Guideline. In one embodiment, the patient is suffering from ICP inhibitor-induced diarrhoea, colitis or enterocolitis with grade 1 toxicities or higher. In another embodiment, the patient is suffering from ICP inhibitor-induced diarrhoea, colitis or enterocolitis with grade 2 toxicities or higher. In yet another embodiment, the patient is suffering from ICP inhibitor-induced diarrhoea, colitis or enterocolitis with grade 3 toxicities or higher. In a further embodiment, the patient is suffering from ICP inhibitor-induced diarrhoea, colitis or enterocolitis with grade 4 toxicities.

According to a preferred embodiment, the inventive composition is for use as a first-line treatment of a patient with ICP inhibitor-induced diarrhoea, colitis or enterocolitis, instead of a steroidal immunosuppressant like a systemic corticosteroid. In one embodiment the patient is not being treated and has not been treated with a corticosteroid.

In an alternative embodiment, the patient first receives a steroidal immunosuppressant (e.g. corticosteroid, like prednisone, prednisolone, methylprednisolone, dexamethasone or budesonide), and only when the ICP inhibitor-induced diarrhoea, colitis or enterocolitis is refractory to steroid treatment, is the composition of the present invention administered to the patient. As used herein, the terms “refractory to steroid treatment” and “steroid refractory diarrhoea, colitis or enterocolitis” refer to diarrhoea, colitis or enterocolitis induced by one or more ICP inhibitors that is unresponsive to steroid therapy. Thus according to a specific alternative embodiment, the inventive composition is for use as second-line treatment in a patient with steroid-refractory IPC inhibitor-induced diarrhoea, colitis or enterocolitis.

In a particular embodiment the pharmaceutical composition of the present invention is administered in a maintenance therapy. That is, the composition is administered to prevent a recurrence or relapse of the adverse event (diarrhoea, colitis and/or enterocolitis). If the adverse event has been successfully treated, the composition may be continued to be administered to prevent a recurrence or relapse of the adverse event. The maintenance dose of the active agent may be lower than the dose used for treating the adverse event.

The present invention in a second aspect, furthermore, relates to a pharmaceutical composition comprising an active agent selected from the group consisting of antibodies specific to TNFα and functional fragments or derivatives thereof, for use in the topical treatment in the ileum and/or the large intestine of a patient, as a prophylactic therapy for ICP inhibitor-induced diarrhoea, colitis or enterocolitis. With regard to the terms “antibodies specific to TNFα and functional fragments or derivatives thereof”, “topical treatment in the ileum and/or the large intestine” and “ICP inhibitor-induced diarrhoea, colitis or enterocolitis” it is referred to the detailed description above.

The term “prophylactic therapy”, as used in the context of the present invention, refers to the administration of an active agent as a preventive therapy. The term “prophylactic therapy for ICP inhibitor-induced diarrhoea, colitis or enterocolitis”, as used in the context of the present invention, refers to the administration of the inventive composition before the onset of symptoms associated with ICP inhibitor-induced diarrhoea, colitis or enterocolitis, as a preventive or semi-preventive therapy.

The inventive composition for use as a topical treatment in the ileum and/or the large intestine of a patient, as a prophylactic therapy allows for systemic treatment with one or more ICP inhibitors at the same time. “At the same time” in the context of the present invention, refers to a therapeutic regimen, where the patient is undergoing concurrent treatment with one or more ICP inhibitors.

In a preferred embodiment of the present invention, the patient is at the same time undergoing treatment with one or more ICP inhibitors. In another preferred embodiment, the patient is at the same time undergoing systemic treatment with one or more ICP inhibitors. For this, the one or more ICP inhibitors may be administered as part of a separate composition at the same time and with the same dosage frequency as the inventive composition or at different times and/or with a different dosage frequency. Moreover, the one or more ICP inhibitors may be administered via the same route of administration or via a different route of administration. With regard to the route of administration of ICP inhibitors it is referred to the description of ICP inhibitors below.

For example, the one or more ICP inhibitors may be administered intravenously or via subcutaneous or intramuscular injection, while the inventive composition is administered orally or rectally. Alternatively, both the one or more ICP inhibitor and the inventive composition may be administered orally or rectally. In case of administration of the one or more ICP inhibitors and the inventive composition via the same route of administration and with the same dosing frequency, the one or more ICP inhibitors may be added to the inventive composition as an additional active agent.

By administration of the inventive composition as a prophylactic therapy, the onset of symptoms associated ICP inhibitor-induced diarrhoea, colitis or enterocolitis can be delayed and/or minimized, or even prevented. Therefore, in one embodiment, the inventive composition for use in the topical treatment in the ileum and/or the large intestine of a patient, as a prophylactic therapy for ICP inhibitor-induced diarrhoea, colitis or enterocolitis, prevents or minimizes and/or slows down symptoms associated with ICP inhibitor-induced diarrhoea, colitis or enterocolitis. In a preferred embodiment the patient is suffering from ICP inhibitor-induced diarrhoea, and the onset of symptoms associated ICP inhibitor-induced colitis or enterocolitis can be delayed and/or minimized, or even prevented. Therefore, the inventive composition for use in the topical treatment in the ileum and/or the large intestine of a patient, as a prophylactic therapy for ICP inhibitor-induced colitis or enterocolitis, prevents or minimizes and/or slows down symptoms associated with ICP inhibitor-induced colitis or enterocolitis.

In another embodiment the inventive composition for use in the topical treatment in the ileum and/or the large intestine of a patient, as a prophylactic therapy for ICP inhibitor-induced diarrhoea, colitis or enterocolitis prevents the onset of ICP inhibitor-induced diarrhoea, colitis or enterocolitis. In yet another embodiment the inventive composition for use in the topical treatment in the ileum and/or the large intestine of a patient, as a prophylactic therapy for ICP inhibitor-induced diarrhoea, colitis or enterocolitis prevents the onset of one or more symptoms associated with ICP inhibitor-induced diarrhoea, colitis or enterocolitis.

In an alternative embodiment the inventive composition for use in the topical treatment in the ileum and/or the large intestine of a patient, as a prophylactic therapy for ICP inhibitor-induced diarrhoea, colitis or enterocolitis delays the onset and/or minimizes the severity of one or more symptoms associated with ICP inhibitor-induced diarrhoea, colitis or enterocolitis. According to a specific embodiment, the topical treatment prevents the development of grade 3 toxicities, preferably grade 2 toxicities, more preferably grade 1 toxicities, most preferably any symptoms, associated with IPC inhibitor-induced diarrhoea, colitis or enterocolitis, in the patient.

The use of the compositions of the present invention for the topical treatment in the ileum and/or large intestine in a patient with ICP inhibitor-induced diarrhoea, colitis or enterocolitis or for prophylactic therapy of ICP inhibitor-induced diarrhoea, colitis or enterocolitis, instead of standard treatment regimen recommended e.g. by the ASCO Practice Guideline, decreases the incidence and/or severity of symptoms of ICP inhibitor-induced diarrhoea, colitis or enterocolitis associated with the use of standard treatment regimen for ICP inhibitor-induced diarrhoea, colitis or enterocolitis.

In a particular embodiment, the treatment of the present invention is associated with, or leads to, an increased Overall Survival of the patients, relative to patients not treated in accordance with the present invention. Preferably, the treatment of the present invention is associated with, or leads to, an increased Overall Survival of the patients, relative to patients treated with corticosteroids. The treatment of the present invention may be associated with, or lead to, an increased Overall Survival of the patients, relative to patients neither treated in accordance with the present invention nor treated with corticosteroids.

The present invention is beneficial in that administration of corticosteroids may be unnecessary. Corticosteroids often lead to an increased infection rate which in turn requires administration of antibiotics. The present invention allows avoiding corticosteroids and antibiotics. Therefore, in a preferred embodiment the composition of the present invention is administered after the first signs of enterocolitis, colitis and/or diarrhoea have been detected.

The present invention further provides a prophylactic use of the compositions described herein. In another embodiment the composition of the present invention is therefore administered before signs of enterocolitis, colitis and/or diarrhoea are detectable, or have been detected. In another embodiment the composition of the present invention is administered to a patient, preferably a cancer patient, more preferably a cancer patient that is being treated with at least one ICP inhibitor, wherein said patient has not been diagnosed as having enterocolitis, colitis and/or diarrhoea. In another embodiment the composition of the present invention is administered to a patient, preferably a cancer patient, more preferably a cancer patient that is being treated with at least one ICP inhibitor, wherein said patient does not suffer from enterocolitis; and/or wherein said patient does not suffer from colitis; and/or wherein said patient does not suffer from diarrhea. Said patient may be is at risk of developing enterocolitis, colitis and/or diarrhoea. Said patient may be suspected of developing enterocolitis, colitis and/or diarrhoea. The present invention relates to the use of the compositions described herein for preventing the onset of, or the development of enterocolitis, colitis and/or diarrhoea in a patient, preferably a cancer patient, more preferably a cancer patient that is being treated with at least one ICP inhibitor.

In a specific embodiment the administration of the active agent improves the efficacy of the treatment with the ICP inhibitor.

ICP inhibitors may be administered to a patient to treat diseases like cancer or to prevent the recurrence of such diseases. Such treatment results in ICP inhibitor-induced adverse events at some point in the majority of cases, with ICP inhibitor-induced adverse events in the GI tract, like inhibitor-induced diarrhoea, colitis or enterocolitis, among the most frequent adverse events.

A “cancer” refers to a broad group of diseases characterized by the uncontrolled growth of abnormal, usually endogenous, cells in the body. Unregulated cell division and growth results in the formation of malignant tumours that invade neighbouring tissues and may also metastasize to distant parts of the body through the lymphatic system or bloodstream. The terms, “cancer,” “tumour,” and “neoplasm,” are used interchangeably herein.

Cancers, which can be treated by ICP inhibitors, are not particularly limited. Exemplary cancers, which can be treated by ICP inhibitors include without limitation: melanoma; lymphoma, like classical Hodgkin lymphoma; glioma; urothelial carcinoma; renal cancer; head and neck squamous cell carcinoma prostate cancer; breast cancer; colon cancer; and lung cancer. According to one embodiment of the present invention, the one or more ICP inhibitor is used to treat a cancer selected from the group consisting of bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the oesophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukaemia, acute myeloid leukaemia, chronic myeloid leukaemia, acute lymphoblastic leukaemia, chronic lymphocytic leukaemia, solid tumours of childhood, lymphocytic lymphoma, cancer of the bladder, cancer of the kidney or ureter, carcinoma of the renal pelvis, neoplasm of the central nervous system (CNS), primary CNS lymphoma, tumour angiogenesis, spinal axis tumour, brain stem glioma, pituitary adenoma, Kaposi's sarcoma, epidermoid cancer, squamous cell cancer, T-cell lymphoma, environmentally induced cancers including those induced by asbestos, and combinations of said cancers.

ICP inhibition, e.g. by inhibition (blockage) of components of the ICP, can be performed against various ICP components, including for example PD-1, PD-L1, CTLA-4, LAG-3, ADAR1 and TIM-3 and combinations of such ICP components. An ICP component may be, without limitation, for example a receptor or a ligand on tumour cells or immune cells such as T-cells, monocytes, microglia, and macrophages. The ICP inhibitors need not be antibodies, but can be also be small molecules or other polymers. If an ICP inhibitor is an antibody, it can be a polyclonal, monoclonal, fragment, single chain, or other antibody variant construct. ICP inhibitors include for example anti-PD-1 antibody, anti-PD-L1 antibody, anti-CTLA4 antibody, anti-LAG-3 antibody and anti-TIM-3 antibody.

ICP inhibitors may target any component of the ICP known in the art that results in the stimulation of the immune system, including but not limited to CTLA-4, PD-1, PD-L1, PDL2, B7-H3, B7-H4, BTLA, HVEM, TIM3, GAL9, LAG3, CSF-1R, VISTA, KIR, 2B4, CD160, CGEN-15049, CHK1, CHK2, A2aR, ADAR1, TIGIT, CD47, ICOS and the B-7 family of ligands. Combinations of inhibitors for a single target component of the ICP or different inhibitors for different target components of the ICP may be used.

ICP inhibitors may be administered at the same time, before, or after the pharmaceutical composition of the present invention. ICP inhibitors may be administered by any appropriate means known in the art for the particular inhibitor, allowing treatment of a specific cancer or infectious disease. Usually ICP inhibitors are administered systemically. Appropriate means known in the art for administering ICP inhibitors include for example intravenous, oral, intraperitoneal, sublingual, intrathecal, intracavitary, intramuscular, and subcutaneous administration.

Preferred routes of administration of the one or more ICP inhibitors include intravenous, intramuscular, intradermal, intraperitoneal, subcutaneous, spinal or other parenteral routes of administration, for example by injection or infusion. The term “parenteral administration” as used herein refers to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In a more preferred embodiment of the present invention, the one or more ICP inhibitors are administered intravenously. Alternatively, the one or more ICP inhibitors may be administered via a non-parenteral route, such as a topical, epidermal or mucosal route of administration, for example, intranasally, orally, vaginally, rectally, sublingually or topically.

Currently available ICP inhibitors include antibodies specific to CTLA-4, PD-1, and PD-L1. According to one embodiment of the present invention the one or more ICP inhibitors selected from the group consisting of antibodies specific to CTLA-4, antibodies specific to PD-1 and antibodies specific to PD-L1. Monoclonal antibodies that are specific to, and thus target, either PD-1 or PD-L1 can block the binding between these two ICP components and boost the immune response against cancer cells. These drugs have shown considerable promise in treating a range of cancer types.

Examples of antibodies specific to PD-1, which are currently used as ICP inhibitors in cancer therapy, include the monoclonal antibodies pembrolizumab (Keytruda®) and nivolumab) (Opdivo®). These antibodies have been shown to be effective in treating several types of cancer, including melanoma of the skin, non-small cell lung cancer, kidney cancer, bladder cancer, head and neck cancers, and Hodgkin lymphoma.

Examples of antibodies specific to PD-L1, which are currently used as ICP inhibitors in cancer therapy, include the monoclonal antibodies atezolizumab (Tecentriq®), avelumab (Bavencio®) and durvalumab (Imfinzi®). These antibodies have also been shown to be effective in treating different types of cancer, including bladder cancer, non-small cell lung cancer, and Merkel cell skin cancer (Merkel cell carcinoma).

CTLA-4 is another B7-CD28 family member that inhibits T-cell functions. It is constitutively expressed by regulatory T-cells but can also be upregulated by other T-cell types, especially CD4+ T-cells, upon activation. CTLA-4 mediates immunosuppression by indirectly diminishing signalling through the co-stimulatory receptor CD28. CTLA-4 signalling has been shown to dampen immune responses against infections and tumour cells (Curran M A et al., Proc Natl Acad Sci USA. 2010 Mar. 2; 107(9):4275-80). Examples of antibodies specific to CTLA-4, which are currently used or being studied as ICP inhibitors in cancer therapy, include the monoclonal antibodies ipilimumab (Yervoy®) and tremelimumab (Pfizer), respectively.

According to one embodiment of the present invention, the one or more ICP inhibitors are a combination of antibodies specific to PD-1 and/or PD-L1 and antibodies specific to CTLA-4, preferably a combination of antibodies specific to PD-1 or PD-L1 and antibodies specific to CTLA-4. According to another embodiment of the present invention, the antibodies specific to PD-1 are selected from the group consisting of pembrolizumab and nivolumab, the antibodies specific to PD-L1 are selected from the group consisting of atezolizumab, avelumab and durvalumab and the antibodies specific to CTLA-4 are selected from the group consisting of ipilimumab and tremelimumab. According to another embodiment of the present invention, the patient to be treated with inventive composition suffers from ICP inhibitor-induced diarrhoea, colitis or enterocolitis, caused by treatment with any one or more of the ICP inhibitors listed above.

For administration of antibodies specific to CTLA-4, PD-1 and PD-L1, for example dosage ranges from about 0.0001 to 100 mg/kg, preferably 0.01 to 5 mg/kg, of the body weight of the patient are adequate. Exemplary dosages can be 0.3 mg/kg body weight, 1 mg/kg body weight, 3 mg/kg body weight, 5 mg/kg body weight or 10 mg/kg body weight or within the range of 1-10 mg/kg body weight. Antibodies are usually administered on multiple occasions. An exemplary dosage regime entails administration once per week, once every two weeks, once every three weeks, once every four weeks, once a month, once every 3 months, once every three to six months or once every 6 months. Intervals can also be irregular as indicated by measuring blood levels of antibody to the target antigen in the patient. In some cases, dosage is adjusted to achieve a specific plasma antibody concentration, e.g. about 1-1000 μg/ml or about 25-300 μg/ml.

Preferred dosage regimens for an CTLA-4, PD-1 or PD-L1 specific antibody include, without limitation, about 1 to 10 mg/kg body weight via intravenous administration, with one antibody or a combination of antibodies being given (i) every four weeks for six dosages, then every three months; (ii) every three weeks; (iii) every two weeks; or (iv) every week.

As used herein, “about” means within an acceptable error range for the particular value as determined by a person skilled in the art, which will depend in part on how the value is measured or determined, i.e. the limitations of the measurement system. For example, “about” can mean within one or more than one standard deviation per the practice in the art. Alternatively, “about” can mean a range of up to 20%. Furthermore, particularly with respect to biological systems or processes, the term can mean up to an order of magnitude or up to 5-fold of a value.

In some cases, two or more CTLA-4, PD-1 and/or PD-L1 specific antibodies are administered simultaneously, in which case the dosage of each antibody administered may fall within the dosage ranges indicated above. If more than one CTLA-4, PD-1 or PD-L1 specific antibody is administered, they may have different binding specificities for CTLA-4, PD-1 or PD-L1, respectively.

Alternatively, ICP inhibitory antibodies can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies.

ADAR1 is an RNA-editing enzyme which catalyzes the hydrolytic deamination of adenosine to inosine in double-stranded RNA (UniProt No. P55265). ADAR1 function can be inhibited by antibodies and functional fragments thereof directed against ADAR1, or by gene silencing techniques, e.g. using siRNA.

In a patient undergoing treatment with one or more ICP inhibitors, the one or more ICP inhibitors may modulate an immune response in the patient, and/or it may inhibit the growth of tumour cells in the patient.

The patient to be treated with the inventive composition may be a cancer patient. In one embodiment, the patient is a cancer patient, and is undergoing or has been undergoing treatment with one or more ICP inhibitors, preferably with one or more ICP inhibitors listed above. Preferably the cancer patient is undergoing treatment with one or more ICP inhibitors. Preferably, treatment with one or more ICP inhibitors is a systemic treatment.

In certain embodiments the patient is undergoing treatment with a cancer vaccine. For example, the patient may receive a combination treatment with an ICP inhibitor and a cancer vaccine.

As used herein, the term “cancer vaccine” has its general meaning in the art and refers to a composition capable of inducing active immunity against at least one cancer antigen. Cancer vaccines typically comprise a source of cancer-associated material or cells (antigen) that may be autologous or allogenic to the subject, along with other components (e.g., adjuvants) to further stimulate and boost the immune response against the antigen. Cancer vaccines can result in stimulating the immune system of the subject to produce antibodies to one or several specific antigens, and/or to produce killer T cells to attack cancer cells that have those antigens. The cancer vaccine can result in a production of antibodies or simply in the activation of certain cells, in particular antigen-presenting cells, T lymphocytes (in particular T-CD8+ cells) and B lymphocytes. The cancer vaccine can be a composition for prophylactic purposes or for therapeutic purposes or both.

There are multiple types of cancer vaccines. Non-limiting examples of cancer vaccines include tumor cell vaccines, antigen vaccines, dendritic cell vaccines, DNA vaccines, and vector based vaccines.

Typically, the cancer vaccine of the present invention comprises a tumor-associated antigen or nucleic acid sequence (e.g. DNA) that encodes for a tumor-associated antigen. Numerous tumor-associated antigens are known in the art. Exemplary tumor-associated antigens include, but are not limited to, 5 alpha reductase, alpha-fetoprotein, AM-1, APC, April, BAGE, beta-catenin, Bell 2, bcr-abl, CA-125, CASP-8/FLICE, Cathepsins, CD 19, CD20, CD21, CD23, CD22, CD33 CD35, CD44, CD45, CD46, CD5, CD52, CD55, CD59, CDCl₂7, CDK4, CEA, c-myc, Cox-2, DCC, DcR3, E6/E7, CGFR, EMBP, Dna78, farnesyl transferase, FGF8b, FGF8a, FLK-I/KDR, folic acid receptor, G250, GAGE-family, gastrin 17, gastrin-releasing hormone, GD2/GD3/GM2, GnRH, GnTV, GP1, hCG, heparanase, Her2/neu, HMTV, Hsp70, hTERT, IGFR1, IL-13R, iNOS, Ki67, KIAA0205, K-ras, H-ras, N-ras, KSA, LKLR-FUT, MAGE-family, mammaglobin, MAP 17, melan-A/MART-1, mesothelin, MIC A B, MT-MMPs, mucin, NY-ESO-1, osteonectin, P170/MDR1, p53, p97/melanotransferrin, PAI-1, PDGF, uPA, PRAME, probasin, progenipoientin, PSA, PSM, RAGE-1, Rb, RCAS1, SART-1, SSX-family, STAT3, STn, TAG-72, TGF-alpha, TGF-beta, Thymosin-beta-15, TNF-alpha, TYRP-, TYRP-2, tyrosinase, VEGF, and glutathione-S-transferase.

In some embodiments, the vaccine is a DNA vaccine. Vectors can be engineered to contain specific DNAs that can be injected into a subject which leads to the DNA being taken up by cells. Once the cells take up the DNA, the DNA will program the cells to make specific antigens, which can then provoke the desired immune response.

In some embodiments, the vaccine consists of a recombinant virus that encodes or expresses a cancer antigen. In some embodiments, the recombinant virus is a poxvirus expressing a tumor antigen and more particularly an orthopoxvirus such as, but not limited to, a vaccinia virus, a Modified Vaccinia Ankara (MVA) virus, or MVA-BN.

In some embodiments, the vaccine composition comprises at least one population of antigen presenting cells that present the selected antigen. The antigen-presenting cell (or stimulator cell) typically has an MHC class I or II molecule on its surface, and in one embodiment is substantially incapable of itself loading the MHC class I or II molecule with the selected antigen.

Preferably, the antigen presenting cells are dendritic cells. Suitably, the dendritic cells are autologous dendritic cells that are pulsed with the antigen of interest (e.g. a peptide). T-cell therapy using autologous dendritic cells pulsed with peptides from a tumor associated antigen is disclosed in Murphy et al. (1996) The Prostate 29, 371-380 and Tjua et al. (1997) The Prostate 32, 272-278. Thus, in some embodiments, the vaccine composition containing at least one antigen presenting cell is pulsed or loaded with one or more antigenic peptides. As an alternative the antigen presenting cell comprises an expression construct encoding an antigenic peptide. The polynucleotide may be any suitable polynucleotide and it is preferred that it is capable of transducing the dendritic cell, thus resulting in the presentation of a peptide and induction of an immune response.

In some embodiments, the vaccine composition includes one or more adjuvants. Adjuvants are substances that non-specifically enhance or potentiate the immune response (e.g., immune responses mediated by CD8-positive T cells and helper-T cells to an antigen, and would thus be considered useful in the medicament of the present invention.

In a specific embodiment, the patient is a cancer patient with a cancer selected from the group consisting of melanoma; lymphoma, like classical Hodgkin lymphoma; glioma; urothelial carcinoma; renal cancer; head and neck squamous cell carcinoma prostate cancer; breast cancer; colon cancer; and lung cancer; and combinations thereof. In another specific embodiment, the cancer is selected from the group consisting of bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the oesophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukaemia, acute myeloid leukaemia, chronic myeloid leukaemia, acute lymphoblastic leukaemia, chronic lymphocytic leukaemia, solid tumours of childhood, lymphocytic lymphoma, cancer of the bladder, cancer of the kidney or ureter, carcinoma of the renal pelvis, neoplasm of the central nervous system (CNS), primary CNS lymphoma, tumour angiogenesis, spinal axis tumour, brain stem glioma, pituitary adenoma, Kaposi's sarcoma, epidermoid cancer, squamous cell cancer, T-cell lymphoma, environmentally induced cancers including those induced by asbestos, and combinations of said cancers.

According to the present invention, the inventive compositions may be in any form that upon administration allows topical treatment in the lumen of the ileum and/or the large intestine of a patient. The composition may be a solid dosage form in the form of pellets, granules, micro particles, nano particles, mini tablets, capsules or tablets and the like. It is known in the art how to manufacture solid dosage forms, for example it can be referred to “Aulton's Pharmaceutics: The Design and Manufacture of Medicines”, Churchill Livingstone title, 4th revised edition, 2013 (ISBN: 978-0-7020-4290-4). Specific example of methods for preparing solid dosage forms comprising antibodies and functional fragments or derivatives include those disclosed in any of the international patent applications PCT/EP2018/074521, PCT/EP2018/074520 and PCT/EP2018/074524.

According to one embodiment, the inventive compositions comprise at least one additive. Suitable additives include hydrophilic polymers, fillers, hydrophilic binders, disintegrants, anti-tacking agents, surfactants, stabilizers, protease resistance enhancers, plasticizers, coalescence agents, lubricants, buffer agents, acidifiers and/or complexing agents. Suitable hydrophilic polymers, fillers, hydrophilic binders, disintegrants, anti-tacking agents, surfactants, plasticizers, coalescence agents, lubricants, buffer agents and/or acidifiers are known to those of skill in the art.

In one embodiment of the present invention, the composition for use in the topical treatment in the ileum and/or the large intestine is administered orally. Oral administration in context of the present invention means the introduction of the composition into gastrointestinal tract via the mouth. In a preferred embodiment of the present invention the composition is a solid dosage form, preferably in the form of a pellet, granule, micro particle, nano particle, mini tablet, sphere, capsule, tablet or multiparticulate drug delivery system coated with a delayed release coating. The term “delayed release”, as used herein, means that the release of the antibody or functional fragment or derivative thereof before the ileum, before the terminal ileum, before the ileocolonic region, or before the colon of the intestine is prevented. The ileocolonic region is the region of the gastrointestinal tract where the small intestine merges with the large intestine, i.e. the terminal ileum. In another embodiment, the composition is a delayed release solid dosage form, preferably in the form of a pellet, granule, micro particle, nano particle, mini tablet, sphere, capsule, tablet or multiparticulate drug delivery system which comprises a sustained release coating or a sustained release matrix. Generally, the term “sustained release” as used herein refers to a time-dependent release, i.e. the antibody or functional fragment or derivative thereof is released over a prolonged period of time, e.g. over at least 6 h, preferably at least 8h, at least 10 h, at least 12 h, at least 14 h, at least 16 h, at least 18 h, or at least 24 h, etc.

In a preferred embodiment, the composition comprises a sustained release core and a delayed release coating. In this embodiment, the composition typically does not comprise a sustained release coating.

Preferably the sustained release core comprises at least one sustained release agent, selected from the group consisting of nonionic poly(ethylene oxide) polymers with a molecular weight between 100,000 and 7,000,000, HPMC 2208 type with a viscosity at 2 wt.-% in water at 20° C. between 3 and 100,000 mPa·s, preferably about 2,308 and 9,030 mPa·s, more preferably 2,663-4,970 mPa·s, xanthan gum, guar gum, tragacanth gum, locust bean gum, acacia gum, chitosan, carbomers, ethylcellulose, polyvinyl acetate, glyceryl (di)behenate, glyceryl palmitostearate, polymethacrylates such as poly(ethyl acrylate, methyl methacrylate, trimethylammonioethyl methacrylate chloride) 1:2:0.1, poly(ethyl acrylate, methyl methacrylate, trimethylammonioethyl methacrylate chloride) 1:2:0.2, or poly(ethylacrylate, methylmethacrylate) 2:1, and combinations thereof.

In another preferred embodiment, the composition comprises an immediate release core, a sustained release coating and a delayed release coating. Typically, the sustained release coating is between the core and the delayed release coating.

In yet another embodiment the composition comprises an immediate release core and a delayed release coating, but no sustained release coating.

Coating materials for the delayed release of a solid dosage form, in particular for targeted release in the ileum or the large intestine, upon oral administration are known in the art. They can be subdivided into coating materials that disintegrate above a specific pH, coating materials that disintegrate after a specific residence time in the gastrointestinal tract and coating materials that disintegrate due to enzymatic triggers specific to the microflora of a specific region of the intestines. Coating materials of these three different categories for targeting to the large intestine have been reviewed for example in Bansal et al. (Polim. Med. 2014, 44, 2, 109-118). These uses of such coating materials have also been described for example in WO 2007/122374 A2, WO 01/76562 A1, WO 03/068196 A1, WO 2008/135090 A1 and GB 2367002 A. In one embodiment of the present invention the delayed release coating comprises at least one component selected from coating materials that disintegrate pH-dependently, coating materials that disintegrate time-dependently, coating materials that disintegrate due to enzymatic triggers in the large intestinal environment, and combinations thereof.

Preferred coating materials among coating materials that disintegrate pH-dependently are selected from poly vinyl acetate phthalate, cellulose acetate trimellitate, hydroxypropyl methylcellulose phthalate HP-50, HP-55 or HP-55S, cellulose acetate phthalate, hydroxypropyl methylcellulose acetate succinate (HPMCAS), poly(methacrylic acid, ethyl acrylate) 1:1 (Eudragit® L100-55, Eudragit® L30D-55), poly(methacrylic acid, methyl methacrylate) 1:1 (Eudragit® L-100, Eudragit® L12.5), poly(methacrylic acid, methyl methacrylate) 1:2 (Eudragit® S-100, Eudragit® S12,5, Eudragit® FS30D), and combinations thereof. Preferred coating materials among coating materials that disintegrate time-dependently are selected poly(ethyl acrylate, methyl methacrylate) 2:1 (e.g. Eudragit® NM 30D, or Eudragit NE 30D); poly(ethyl acrylate, methyl methacrylate, trimethylammonioethyl methacrylate chloride) 1:2:0.1 (e.g. Eudragit® RS 30D); ethylcellulose (e.g. Surelease® or Aquacoat ECD); poly(ethyl acrylate, methyl methacrylate, trimethylammonioethyl methacrylate chloride) 1:2:0.2 (e.g. Eudragit® RL 30D); polyvinyl acetate (eg. Kollicoat® SR 30D); and combinations thereof. Preferred coating materials among coating materials that disintegrate due to enzymatic triggers in the large intestinal environment are selected from chondroitin sulfate, pectin, guar gum, chitosan, inulin, lactulose, raffinose, stachyose, alginate, dextran, xanthan gum, locust bean gum, arabinogalactan, cyclodextrin, pullulan, carrageenan, scleroglucan, chitin, curdulan, levan, amylopectin, starch, amylose, resistant starch, azo compounds being degraded by azo bonds splitting bacteria, and combinations thereof. The delayed release coating optionally comprises one or more further excipients.

In one embodiment of the present invention the coating material for the delayed release coating comprises one, two, three, etc., component(s) selected from the coating materials that disintegrate pH-dependently, the coating materials that disintegrate time-dependently, and the coating materials that disintegrate due to enzymatic triggers in the intestinal environment, listed above, and combinations thereof. In another embodiment of the present invention, the delayed release coating comprises a combination of at least one coating material that disintegrates pH-dependently and at least one coating material that disintegrates due to enzymatic triggers in the intestinal environment.

For example, a delayed release coating can be designed to focus the delivery of the composition comprising the antibody or functional fragment or derivative thereof entirely in the large intestine, beginning at the cecum, and continuing through the ascending, transverse, and descending colon, and ending in the sigmoid colon. Alternatively, for example, a delayed release coating can be designed to begin the delivery of the antibody or functional fragment or derivative thereof in the ileum and end the release in the transverse colon. The possibilities and combinations are numerous.

In a different embodiment of the present invention, the composition for use in the topical treatment in the ileum and/or the large intestine is administered rectally. Rectal administration in context of the present invention means the introduction of the composition into gastrointestinal tract via the anus. In a preferred embodiment of the present invention the composition is used in the form of an enema, a gel, a foam or a suppository.

A person skilled in the art can readily determine the effective amount of TNFα specific antibody or functional fragment or derivative thereof to be administered as part of the composition for use in accordance with the present invention. In general, an effective amount of a TNFα specific antibody or functional fragment or derivative thereof according to the invention is the lowest amount required to produce a therapeutic effect, i.e., to treat diarrhoea, colitis or enterocolitis induced by one or more ICP inhibitors. The exact amount of a TNFα specific antibody or functional fragment or derivative thereof to be administered to a patient can vary depending on the state and severity of the disorder and the physical condition of the patient.

According to one embodiment of the present invention, the inventive composition provides a therapeutically effective dose of the TNFα specific antibody or functional fragment or derivative thereof in the lumen of the ileum and/or the large intestine of a patient for preventing, minimizing and/or delaying or alleviating symptoms associated with ICP inhibitor-induced diarrhoea, colitis or enterocolitis. A “therapeutically effective dose” is the amount of the at least one TN Fa specific antibody or functional fragment or derivative thereof required to provide the desired therapeutic effect. The exact amount may vary for different antibodies or functional fragments or derivatives thereof and/or for individual patients, but can be determined by one skilled in the art.

A therapeutic effective dose in the lumen of the ileum and/or the large intestine can be achieved e.g. by means of targeting the inventive composition comprising anti-TNFα antibodies or functional fragments or derivatives thereof. The way and means of targeting the inventive composition in the lumen of the ileum and large intestine are not particularly limited and can be achieved by methods known in the art. These include taking advantage of the innate processes of the gastrointestinal tract that result e.g. in differences in pH and microflora and specific residence times of ingested materials in different sections of the GI tract. Methods for sampling concentrations of specific proteins including specific antibodies in the intestinal lumen are known in the art. Samples can be collected for example from expelled faeces, or using a flexible tube inserted via the anus. TNFα antibody concentration can then be determined using ELISA or Western Blots or other immunochemical techniques, similarly to what has been described for the measurement of faecal TNFα concentration in Nicholls et al. (J Clin Pathol. 1993 August; 46(8): 757-760), with an antibody specific to the anti-TNF antibody or functional fragment or derivative thereof used in the composition.

One unit dose of the inventive composition may comprise for example an amount of active agent in the range of from about 0.05 mg to about 1,000 mg, 0.1 mg to about 200 mg, or from about 1 mg to about 100 mg, or from about 10 mg to about 50 mg.

The compositions comprising the TNFα specific antibody or functional fragment or derivative thereof, according to the invention, can be administered to the patient for example once per day, twice per day, or three times per day. In a preferred embodiment, the composition is administered once daily.

| SEQ ID NO: | Description of the amino acid sequence |
|---|---|
| 1 | Light chain of a humanized anti-TNFα antibody |
| | (=light chain of Ab-REW) |
| 2 | Heavy chain of a humanized anti-TNFα antibody |
| 3 | CDR L1 of antibody Ab-REW |
| 4 | CDR L2 of antibody Ab-REW |
| 5 | CDR L3 of antibody Ab-REW |
| 6 | CDR H1 of antibody Ab-REW |
| 7 | CDR H2 of antibody Ab-REW |
| 8 | CDR H3 of antibody Ab-REW |
| 9 | V_{H} of antibody Ab-REW |
| 10 | V_{L} of antibody Ab-REW |
| 11 | Heavy chain of antibody Ab-REW |
| 12 | Light chain of surrogate antibody cV1q-huFc |
| 13 | Heavy chain of surrogate antibody cV1q-huFc |
| 14 | Modified heavy chain of antibody Ab-REW |
| 15 | Heavy chain of antibody Ab-AA |
| 16 | Modified heavy chain of antibody Ab-AA |

### EXAMPLES

### Examples 1 and 2

Materials and Methods Applied in the Examples

Citrate-TRIS buffer pH 7 preparation: A solution of sodium citrate 100 mM (2.942 g and completed to 100.0 mL with purified water) was prepared. A solution of citric acid 100 mM (3.842 g dissolved and diluted to 200.0 ml with purified water) was prepared. The pH of the citric acid solution was adjusted to 3.5 with the sodium citrate solution. A TRIS solution 1M (12.114 g and completed to 100.0 ml with purified water) was prepared. The pH of the citrate buffer was adjusted to pH 7.0 with the TRIS solution.

Preparation of Pellets

Formulation Composition (Pellets Core)

| Example 1 (immediate | | Example 2 (sustained | |
|---|---|---|---|
| release pellet core) - | | release pellets core) | |
| Microcrystalline | 50% * | Microcrystalline | 75% * |
| cellulose | | cellulose | |
| Sorbitol | 45% * | Compritol 888 ATO | 10% * |
| Sodium starch | 5% * | Sorbitol | 15% * |
| glycolate | | | |
| Adalimumab | 0.82% (loading | Adalimumab | 1.08% (loading |
| | achieved) | | achieved) |

* of dry mix prior to the addition of adalimumab

Manufacturing Steps:

Dry mixing: The excipients required for each batch (batch size: 10 g) were mixed using the Mixer attachment (double-paddle mixer) from the Caleva Multilab equipment for about 5 minutes predetermined period of time at 50 rpm.

Wet mixing: After the dry mixing step, an adalimumab solution containing 0.1% w/v polysorbate 20 was slowly added to the powder blend of excipients under mixing and mixed for 10 minutes at 50 rpm

Extrusion: The wet mass was then emptied from the mixer and extruded through 1 mm diameter and 1 mm depth holes of the extrusion die using a screw extruder at a constant speed (150 rpm) until all wet mass is extruded.

Spheronization: The wet extrudate was then fed to the spheronizer attachment, consisting of a grooved plate, which by rotation breaks the wet extrudates into smaller fragments that depending on time, speed and the nature of the individual components of the extrudate become then round (wet spheroids). The extrudate was spheronized for a predetermined amount of time at 1500 rpm.

The wet pellets obtained from the spheronization step were then collected in a disposable weighing boat and dried overnight at 40° C. in a drying cabinet.

Delayed (Enteric) Release Coating

Adalimumab containing pellets (from Example 1 and 2) were coated with an enteric coating containing Eudragit L30D-55. The coating suspension was prepared blending the required amount of Eudragit L30D-55 dispersion with glyceryl monostearate (GMS) emulsion. GMS emulsion was prepared by dissolving polysorbate 80 in water followed by addition of GMS. The mixture was then heated to 75° C. and kept at this temperature for 15 minutes under continuous magnetic stirring. The cooled GMS emulsion was then added to the Eudragit L30D-55 followed by addition of triethyl citrate (plasticizer). The suspension is stirred for 30 minutes prior coating.

The coating suspension was then sprayed on adalimumab containing pellets to reach a target of 30% polymer weight gain. The coating was applied using a MiniGlatt fluid bed coater (bottom spray) equipped with a microkit to allow coating of small size batches. Eudragit L30D-55 suspension was sprayed at an inlet temperature of 40° C., product temperature of 33.0-34.5° C., airflow of 24-28 m³/h and atomizing air pressure of 0.2-0.3 bar.

Dissolution of adalimumab from pellets A quantity of adalimumab loaded pellets was placed in a 5 ml cryo tube and 4.0 ml of buffer was added to yield a nominal 1 mg/ml adalimumab concentration, based on the theoretical calculated adalimumab loading. Citrate-TRIS buffer pH 7 was used as buffer. Samples are agitated during the entire duration of the experiment. Supernatant samples were taken at predetermined time points, centrifuged and the supernatant was analyzed in terms of total protein content. In the case of enteric coated pellets, the pellets were first exposed to 0.1N HCl for 2 hours under continuous agitation and then this fluid was removed and then pH 7.0 citrate-TRIS buffer was added for the buffer stage which was run as described previously.

Total protein content quantification (Bradford): Total protein quantification was done by colorimetry following the Bradford method with a Coomassie Plus assay (Thermo Fisher Scientific). Briefly, 6.6 μl of sample were pipetted to the bottom of a 96-well plate and 200 μl of Coomassie Plus reagent were added and mixed by agitation for 30 s at 500 rpm. The samples were then incubated at room temperature for 10 min after which the absorbance at 595 nm was recorded (Tecan plate reader) and the blank subtracted. Quantification was done using a freshly prepared standard curve.

Results

Release from Adalimumab Immediate and Sustained Release Pellets Cores

Uncoated pellets of Example 1 disintegrated fast in citrate-TRIS pH 7.0 buffer resulting in a fast and almost complete adalimumab release within 1 hour. On the other hand, release of adalimumab from uncoated pellets in Example 2 was sustained over the duration of the experiment.

| | Time (h) | 1 | 2 | 4 | 8 | 24 |
|---|---|---|---|---|---|---|
| % | Example 1 | 85.2 | 89.7 | | | 91.43 |
| Adalimumab | (uncoated | | | | | |
| release | pellets) | | | | | |
| | Example 2 | 25.8 | 34.0 | 44.0 | 53.1 | 61.5 |
| | (uncoated | | | | | |
| | pellets | | | | | |

Release from Adalimumab Delayed (Enteric) Release Pellets Cores

Both batches of adalimumab pellets were coated with a Eudragit L30D-55 dispersion. After 2 hours in 0.1N HCl the coated pellets were completely acid resistant (no release of adalimumab). Upon exchange to citrate-TRIS pH 7.0 buffer (to simulate small intestinal luminal pH), adalimumab release was initiated (release 5%).

### Example 3

The therapeutic efficacy of an anti-TNFα antibody following local administration was investigated in an IBD mouse model. Due to the species-specificity of Ab-REW which does not bind to mouse TNFα a surrogate antibody (cV1q) consisting of the variable domain of the cV1q antibody (Echtenacher et al., 1990) combined with a mouse IgG2a constant region was used. The T-cell transfer model was chosen as the pattern of gene expression in this model most closely reflects altered gene expression in IBD (if compared to DSS- and TNBS-induced colitis) (te Velde et al., Inflamm Bowel Dis 2007; 13(3):325-30). This model is clinically characterized by a progressive body weight loss and soft stool. With respect to histopathology inflammation reaches from the caecum to the rectum with infiltration of macrophages accompanied by moderate numbers of activated CD4+ lymphocytes, mucin depletion and epithelial hyperplasia resulting in glandular elongation and mucosal thickening. Increased gene expression of TNF-α, IFN-γ, IL-6, CCR1, CCR2, CCR5, CXC chemokine receptor 3 and their ligands was described (Nagaoka and Radi, Front Biosci 2012 Jun. 1; 4:1295-314).

Colitis was induced by adoptive transfer of naïve CD4+ T-cells from WT mice to immune-deficient mice, inducing transmural colitis in the recipients. Briefly, T-cells (CD44⁻/CD62L⁺) were harvested and purified from naïve C57BL/6 mice and 0.5×10⁶ cells were intraperitoneally injected into RAG2^{-/-} mice. Engraftment of T-cells was confirmed on Day 20.

A rat/murine chimeric monoclonal antibody of the IgG2a isotype (the mouse homologue to human IgG1) specific for mouse TNFα was used for treatment (Echtenacher et al., J Immunol 1990; 145:3762-66).

The mice were divided into 4 groups (for naïve mice n=5; for all other groups n=12), as follows:
Group 1 were naïve mice.
Group 2 received vehicle intrarectally.
Group 3 received IgG2a intrarectally.
Group 4 received IgG2a intraperitoneally as positive control.

Anti-TNFα treatment started on day 21 after T-cell transfer when colitis had been established in the animals and lasted for 28 days. Animals receiving rectal administration (300 μg/d of IgG2a or 200 μl vehicle) were treated daily while animals injected intraperitoneally with IgG2a were treated twice a week. Endpoints were body weight, endoscopy score (day 14-49), histological score (day 49) and cytokines in the colon (day 49).

Results:

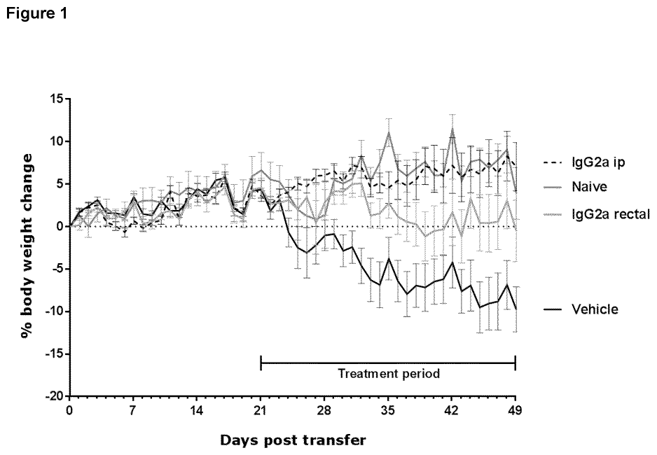

The mice in the naïve and the IgG2a (ip, ir) treated groups maintained or continued to gain weight while the animals in the vehicle treated group steadily lost weight (FIG. 1).

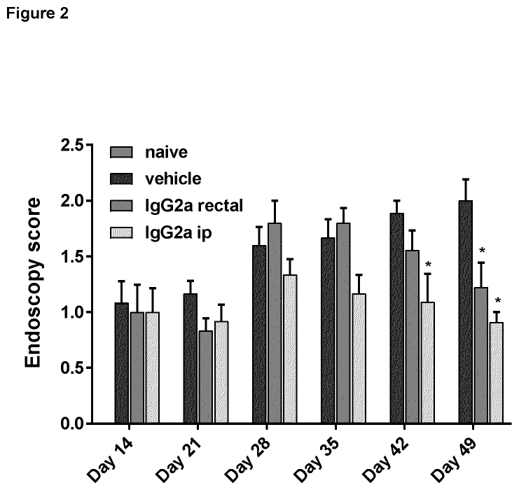

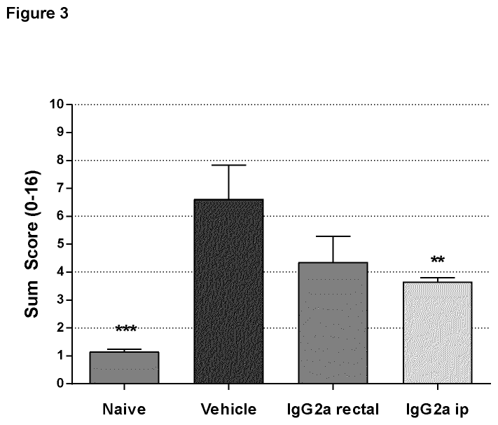

A significant reduction in endoscopy score and a decrease in histological score was observed in IgG2a-treated groups (ir and ip); see FIGS. 2 and 3. During endoscopy colitis was scored on a 0-4 scale (0: normal; 1: loss of vascularity; 2: loss of vascularity and friability; 3: friability and erosions; 4: ulcerations and bleeding). The total histological score is the sum of the individual scores (0-4 each) for inflammation, oedema, goblet cell depletion and epithelial damage.

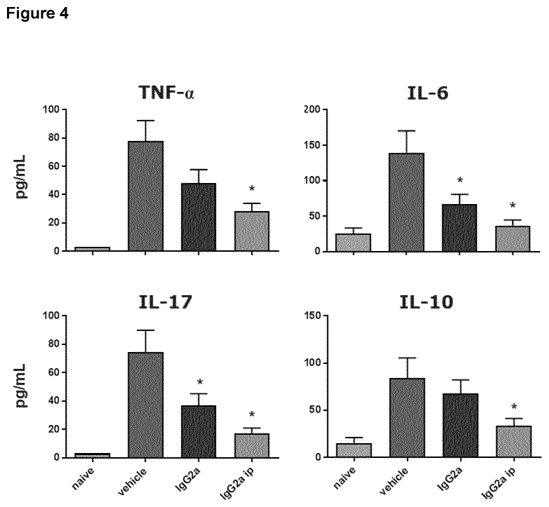

Intraperitoneal treatment with IgG2a resulted in the highest reduction of cytokines in colon followed by intra-rectal treatment with IgG2a. See FIG. 4. Given values are means of data from proximal, medial and distal colon.

In summary, the topical treatment works in the mouse T-cell transfer model as shown by significantly reduced endoscopy score, reduced histological score, reduced cytokines in colon and less body weight loss.

### Example 4

Dose Response Study on the Treatment of Colitis in a Colitis Mouse Model

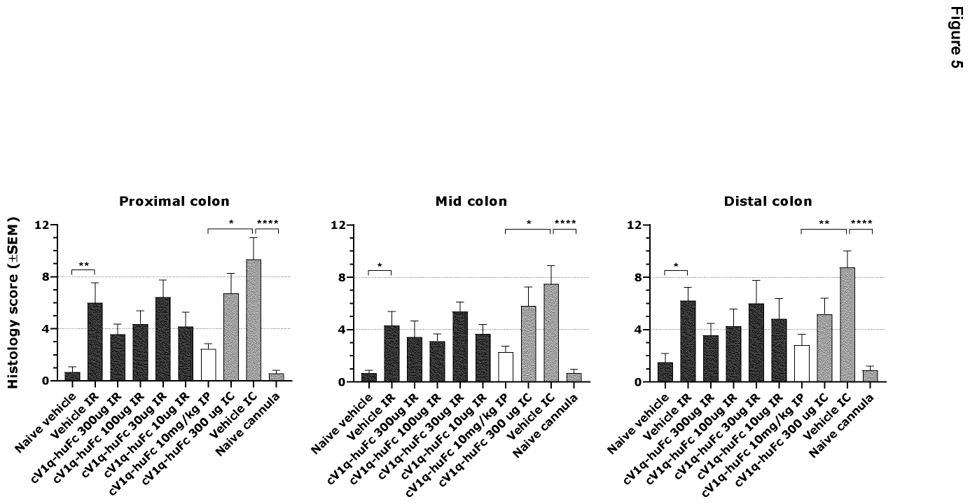

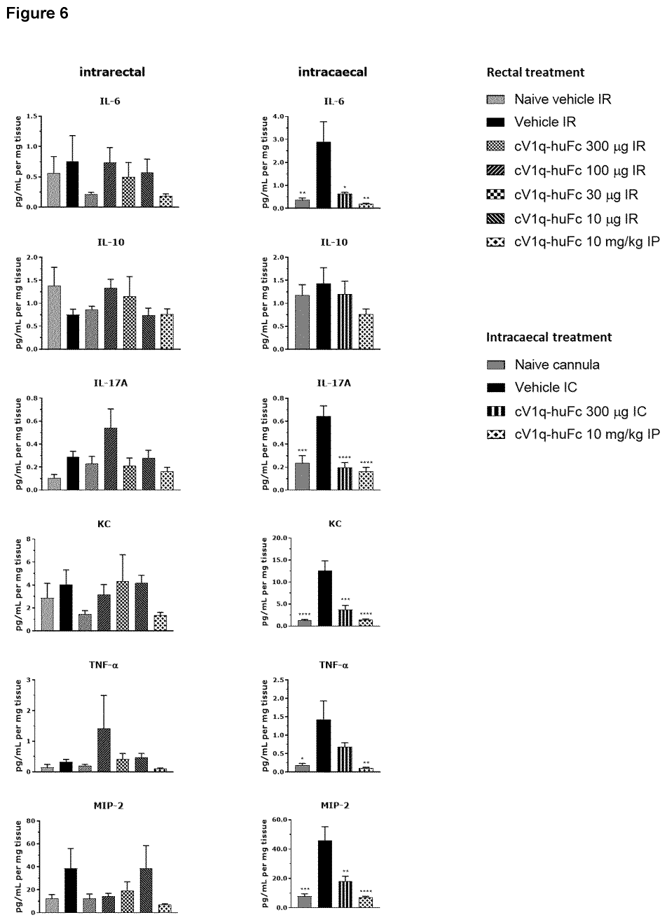

In a dose response study, the therapeutically effective dose of rectally administered cV1q-huFc surrogate antibody was assessed in the T-cell transfer IBD mouse model using Tg32-SCID mice (human FcRn transgenic mice). Due to the species-specificity of Ab-REW which does not bind to mouse TNFα a surrogate antibody (cV1q-huFc) consisting of the variable domain of the cV1q antibody (Echtenacher et al., 1990) and the constant domain of Ab-REW was used. The light and heavy chain sequences of the surrogate antibody are shown in SEQ ID NO:12 and 13, respectively. Colitis was induced by an adoptive transfer of naïve CD4+ T-cells from C57Bl/6 mice into Tg32-SCID mice. Treatment with cV1q-huFc started on day 14 after the adoptive transfer and lasted until day 42. Mice received either daily intra-rectal administrations of cV1q-huFc (10, 30, 100 and 300 μg/mouse), daily intracaecal applications (300 μg/mouse) or twice per week an intraperitoneal injection of 10 mg/kg (control). A dose-dependent improvement in the total histological score, consisting of colonic inflammation (submucosal and muscularis/serosal), crypt damage, erosion, hyperplasia and oedema was observed in the proximal and distal colon after rectal treatment (FIG. 5). Also the intracaecal treatment resulted in a reduced histological score. Further, a reduction in colonic cytokines IL-6, IL-17A, KC, TNFα and MIP-2 were observed after both rectal and intracaecal administration (FIG. 6). This was significant in case of IL-17A, KC and MIP-2 following intracaecal administration. In summary, local administration of a TNFα inhibitor results in reduced inflammation in the colon.

### Example 5

Systemic Exposure Following Rectal Administration

The study was conducted in healthy mice and mice suffering from colitis to determine systemic exposure to cV1q-huFc following rectal administration. Colitis was induced by exposing the Tg32-SCID mice to 3% DSS-treated drinking water from day 0 to day 5. Healthy and diseased mice were rectally treated with a single dose (SD) or multiple doses (MD; 0.1 or 0.3 mg) of cV1 q-huFc. Due to limitations in blood collection, only three blood samples could be drawn from each mouse (left eye, right eye, terminal). Thus, each treatment group (n=10-14 mice) was divided into two subgroups (n=5-7 mice) for blood collections. Single dosed animals received the cV1q-huFc antibody on day 10 when colitis was established and blood samples were collected at 1, 4 and 24 h (subgroup 1) and 2, 8 and 48 h (subgroup 2) after dosing. Mice receiving multiple doses of cV1q-huFc received daily rectal administrations from day 10-14 and blood samples were collected 24, 72 and 120 h (subgroup 1) and 48, 96 and 144 h (subgroup 2) after the first cV1q-huFc dose on day 10, always just before administration of the next dose. In parallel, two groups (healthy and diseased) received a single intravenous injection of cV1q-huFc at 5 mg/kg. Blood samples were collected as in the rectally treated mice receiving a single dose. Plasma samples were analysed by Imperacer® Immuno-PCR.

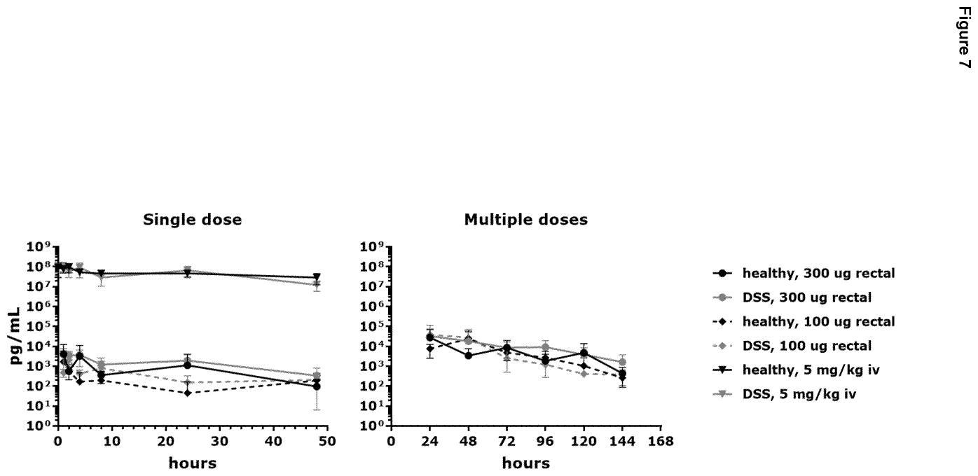

Systemic exposure following rectal administration was very low compared to intravenous administration (FIG. 7). It was slightly but not significantly higher in colitic animals compared to healthy animals. A single dose of rectally applied cV1q-huFc resulted in a calculated systemic bioavailability of <0.01% if dose normalized exposures (AUC) were compared. There was no systemic accumulation observed over time after 5 daily doses.

### Example 6

Systemic Exposure and Tissue Concentrations after Intracaecal Administration

cV1q-huFc tissue concentrations as well as systemic exposure was determined in healthy mice and mice suffering from colitis following intracaecal administration. Male Tg32-SCID mice underwent surgery where a cannula was placed from the caecum to the back between the shoulder blades, exposing the tip of the cannula which was secured in place using suture, wound clips and tissue glue. Animals received Buprenorphine (for 3 d) and Baytril (for 5 d) following surgery and were allowed to recover for 2-4 weeks before being allocated to the study. Colitis was induced by exposing the mice to 3% DSS-treated drinking water from day 0 to day 5. Healthy and diseased mice were treated with a single dose (n=16 per group) or multiple doses (n=8 per group) of cV1q-huFc. Healthy mice received a dose of 0.3 mg/administration while two dose levels (0.1 and 0.3 mg/administration) were tested in colitic mice. When dosing the animals, cV1q-huFc was applied in 0.1 mL via the cannula which was afterwards rinsed with 0.05 mL saline to avoid that any drug substance remained in the cannula and did not reach the caecum. Four mice were sacrificed at each time point and colon and blood (plasma) was collected. Single dosed animals received the cV1q-huFc antibody on day 10 when colitis was established and animals were sacrificed 1, 4, 8 and 12 h after dosing. Mice receiving multiple doses of cV1q-huFc received daily intracaecal administrations from day 10-14 and were sacrificed 24 and 120 h after the first cV1q-huFc dose on day 10. In parallel, two groups with induced colitis received a single or multiple rectal administration of cV1q-huFc at 0.3 mg/application. Blood (plasma) samples were collected as for the intracaecally treated animals. Plasma samples were analysed by Imperacer® Immuno-PCR. Colons were excised at sacrifice, rinsed, weighed and trimmed to 5 cm in length. A 1 cm piece from both the proximal and distal ends was snap frozen for analysis by ELISA.

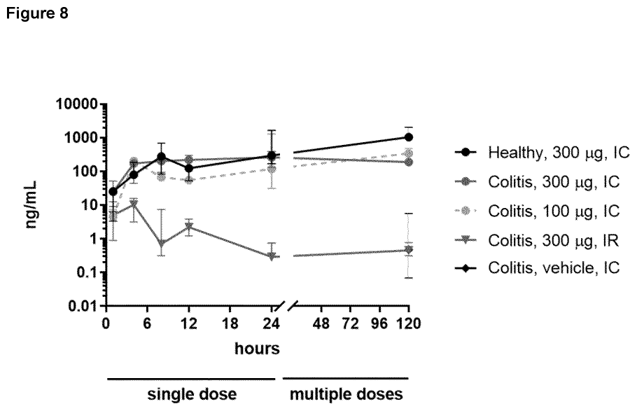

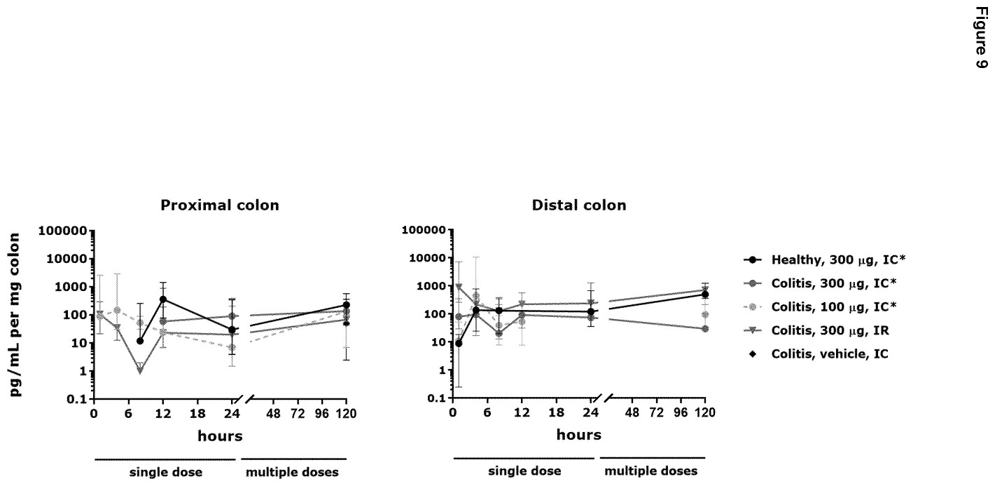

Measured plasma concentrations were highly variable after intracaecal administration, as already observed after rectal administration. Systemic exposure (AUC) was approximately 60-fold (single dose) or 200-fold (multiple doses) increased after intracaecal administration compared to rectal administration, which, however, was still a very low overall systemic exposure (FIG. 8). A slight accumulation of cV1q-huFc in plasma was observed in most groups after 5 daily doses. Similar to the plasma concentrations, cV1q-huFc levels in tissue were highly variable within groups and time points. Both routes of administration, intracaecal and intra-rectal, resulted in measurable antibody concentrations in the proximal as well as in the distal colon (FIG. 9). In contrast to the systemic exposure where a clear difference between rectal and intracaecal treatment was observed, the tissue levels were comparable for both administration routes. No significant difference was observed between healthy and diseased animals. Repeated daily administration on 5 consecutive days resulted in a slight accumulation of cV1q-huFc in the proximal and distal colon in most groups.

### Example 7

Effect on Nivolumab-Stimulated Human CD4⁺ T-Cells

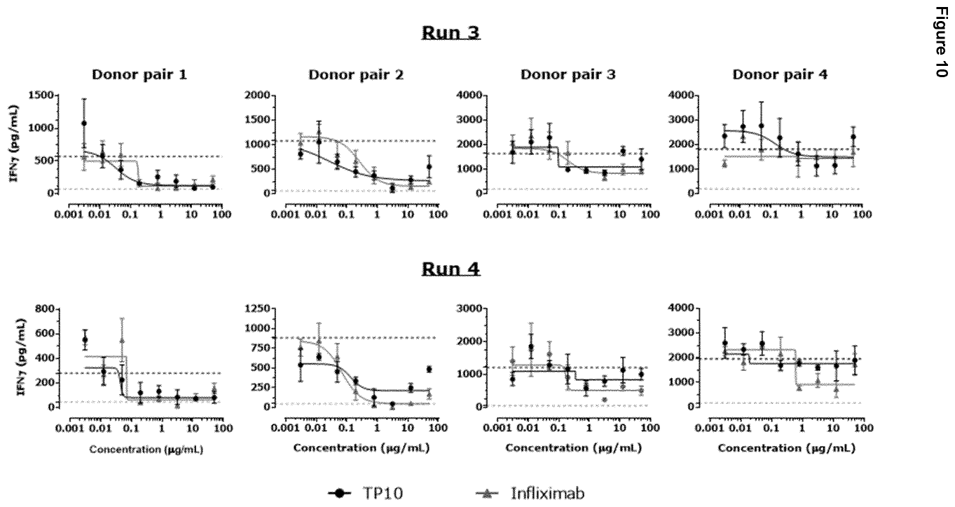

Immune checkpoint inhibitors targeting PD-1 or CTLA-4 are very effective cancer treatments. However, they come with some severe immune-related adverse events such as colitis. Thus, the effect of Ab-REW on nivolumab-stimulated human CD4⁺ T-cells was investigated by measuring the IFNγ secretion in a mixed lymphocyte reaction (MLR). It was shown that nivolumab induced higher levels of IFNγ secretion from CD4⁺ T-cells in comparison to untreated cells. Concomitant incubation with nivolumab and Ab-REW or infliximab inhibited IFNγ secretion in a dose-dependent manner, although quite some variability was observed between the different donor pairs and runs (FIG. 10). However, the trend was consistent across runs and donor pairs, indicating that this is a true biological phenomenon. It appeared that donor pairs with the capability to secrete higher levels of IFNγ did not respond as robustly as donor pairs demonstrating weaker IFNγ secretion, suggesting that donor variability and the strength of the initial stimulation affects the efficacy of anti-TNFα agents in this context. Interestingly, IC₅₀ values were generally lower for Ab-REW (range 0.028-0.266 μg/mL) than for infliximab (range 0.068-0.326 μg/mL), suggesting that Ab-REW is possibly a stronger inhibitor of nivolumab-mediated IFNγ secretion than infliximab.

1. A pharmaceutical composition for use in the treatment or prevention of at least one adverse event of the gastro-intestinal tract induced by a cancer therapy in a cancer patient in need thereof, wherein said pharmaceutical composition comprises an active agent selected from the group consisting of antibodies specific to tumour necrosis factor alpha (TNFα) and functional fragments and derivatives thereof and is formulated for topical administration to an affected part of the gastrointestinal tract of said patient.

2. The pharmaceutical composition according to claim 1, wherein said cancer therapy is characterized by the administration of one or more immune checkpoint (ICP) inhibitors.

3. The pharmaceutical composition according to claim 2, wherein the administration of one or more ICP inhibitors is interrupted for less than four weeks.

4. The pharmaceutical composition according to claim 1, wherein said at least one adverse event is selected from the group consisting of ICP inhibitor-induced diarrhoea, ICP inhibitor-induced colitis, ICP inhibitor-induced enterocolitis and combinations thereof.

5. The pharmaceutical composition according to claim 4, wherein said ICP inhibitor-induced diarrhoea, colitis and/or enterocolitis is characterized by grade 1 toxicity or grade 2 toxicity.

6. The pharmaceutical composition according to claim 4, wherein said topical administration of said pharmaceutical composition prevents progression of said ICP inhibitor-induced diarrhoea, colitis and/or enterocolitis to a higher grade toxicity.

7. The pharmaceutical composition according to claim 1, wherein said topical administration of said pharmaceutical composition constitutes a prophylactic therapy for ICP inhibitor-induced diarrhoea, colitis and/or enterocolitis.

8. The pharmaceutical composition according to claim 7, wherein the patient is undergoing treatment with one or more ICP inhibitors at the same time as said pharmaceutical composition is being topically administered.

9. The pharmaceutical composition according to claim 1, wherein said patient is suffering from ICP inhibitor-induced diarrhoea, but not of ICP inhibitor-induced colitis or ICP inhibitor-induced enterocolitis, and wherein said composition is formulated for use in preventing the development or onset of ICP inhibitor-induced colitis and/or ICP inhibitor-induced enterocolitis in said patient.

10. The pharmaceutical composition according to claim 9, wherein said patient is suffering from ICP inhibitor-induced diarrhoea of grade 2 toxicity.

11. The pharmaceutical composition according to claim 9, wherein said patient is suffering from ICP inhibitor-induced diarrhoea of grade 1 toxicity.

12. The pharmaceutical composition according to claim 1, wherein the one or more ICP inhibitors are selected from the group consisting of antibodies specific to cytotoxic T-lymphocyte-associated protein-4 (CTLA-4), antibodies specific to programmed cell death protein-1 (PD-1) and antibodies specific to programmed death-ligand 1 (PD-L1).

13. The pharmaceutical composition according to claim 12, wherein the antibodies specific to PD-1 are selected from the group consisting of pembrolizumab and nivolumab; the antibodies specific to PD-L1 are selected from the group consisting of atezolizumab, avelumab and durvalumab, whereas the antibodies specific to CTLA-4 are selected from the group consisting of ipilimumab and tremelimumab.

14. The pharmaceutical composition according to claim 1, wherein the antibodies specific to TNFα and functional fragments thereof are selected from the group consisting of infliximab, adalimumab, etanercept, certolizumab pegol, golimumab, and functional fragments and derivatives thereof.

15. The pharmaceutical composition according to claim 1, wherein the amino acid sequence of the antibody specific to TNFα or the functional fragments thereof is selected from the group consisting of:
i) the amino acids 233P, 234V, 235A, and a deletion at amino acid position 236; and the amino acid 434A or the amino acids 252Y, 254T and 256E; and optionally the amino acids 239D, 330L and 332E or the amino acids 326A, 332E and 333A;
ii) the amino acids 380A and 434A, and optionally the amino acid 307T;
iii) the amino acid 434W, and optionally the amino acid 428E and/or the amino acid 311R, wherein the amino acid numbering refers to EU numbering.

16. The pharmaceutical composition according to claim 1, wherein said topical administration comprises oral administration of the pharmaceutical composition to said patient.

17. The pharmaceutical composition according to claim 16, wherein the pharmaceutical composition is a delayed release formulation.

18. The pharmaceutical composition according to claim 16, wherein the pharmaceutical composition is a solid dosage form in the form of a pellet, granule, micro particle, nano particle, mini tablet, sphere, capsule, tablet or multiparticulate drug delivery system, wherein said dosage form is coated with a delayed release coating that prevents release of the active agent before entering the ileum, the ileocolonic region or the large intestine of the gastrointestinal (GI) tract.

19. The pharmaceutical composition according to claim 18, wherein the delayed release coating comprises at least one component selected from the group consisting of poly vinyl acetate phthalate, cellulose acetate trimellitate, hydroxypropyl methylcellulose phthalate HP-50, HP-55 or HP-55S, cellulose acetate phthalate, hydroxypropyl methylcellulose acetate succinate (HPMCAS), poly(methacrylic acid, ethyl acrylate) 1:1, poly(methacrylic acid, methyl methacrylate) 1:1, poly(methacrylic acid, methyl methacrylate) 1:2, chondroitin sulfate, pectin, guar gum, chitosan, inulin, lactulose, raffinose, stachyose, alginate, dextran, xanthan gum, locust bean gum, arabinogalactan, amylose, cyclodextrin, pullulan, carrageenan, scleroglucan, chitin, curdulan, levan, amylopectin, starch, resistant starch, azo compounds being degraded by azo bonds splitting bacteria, and combinations thereof.

20. The pharmaceutical composition according to claim 16, wherein said composition comprises a sustained release coating or a sustained release matrix.

21. The pharmaceutical composition according to claim 20, wherein said sustained release coating or a sustained release matrix comprises a material that disintegrates time-dependently.

22. The pharmaceutical composition according to claim 21, wherein said material that disintegrates time-dependently is selected from the group consisting of a poly(ethyl acrylate, methyl methacrylate) 2:1 such as Eudragit® NM 30D or Eudragit NE 30D; a poly(ethyl acrylate, methyl methacrylate, trimethylammonioethyl methacrylate chloride) 1:2:0.1 such as Eudragit® RS 30D; an ethylcellulose such as Surelease® or Aquacoat ECD; poly(ethyl acrylate, methyl methacrylate, trimethylammonioethyl methacrylate chloride) 1:2:0.2 such as Eudragit® RL 30D; a polyvinyl acetate such as Kollicoat® SR 30D; and combinations thereof.

23. The pharmaceutical composition according to claim 16, wherein said composition comprises a sustained release core coated with a delayed release coating.

24. The pharmaceutical composition according to claim 16, wherein said composition comprises a core, a sustained release coating and a delayed release coating.

25. The pharmaceutical composition according to claim 1, wherein said topical administration comprises a rectal formulation in the form of an enema, a gel, a foam or a suppository.

26. The pharmaceutical composition according to claim 1, wherein the affected part of the gastrointestinal tract of said patient includes the patient's ileum and/or large intestine.

27. The pharmaceutical composition according to claim 3, wherein said cancer therapy characterized by the administration of one or more ICP inhibitors is not interrupted.

28. A method for the treatment or prevention of at least one adverse event of the gastro-intestinal tract that is induced by a cancer therapy characterized by the administration of one or more immune checkpoint (ICP) inhibitors in a patient in need thereof, wherein said method comprises the step of topically administering the pharmaceutical composition according to claim 1 to an affected part of the patient's gastrointestinal tract that includes the ileum and/or the large intestine, further wherein said patient is a cancer patient actively undergoing ICP cancer therapy.
